(19) United States
(12) Patent Application Publication
(10) Pub. No.: US 2006/0166282 A1
(43) Pub. Date: Jul. 27, 2006

(54) Methods and compositions for screening cell binding molecules

(76) Inventors: Sharat Singh, Los Altos, CA (US); Po-Ying Chan-Hui, Mayne Island (CA); Hrair Kirakossian, San Jose, CA (US)

Correspondence Address: MONOGRAM BIOSCIENCES, 345 OYSTER POINT BLVD, SOUTH SAN FRANSISCO, CA, 94080, US

(21) Appl. No.: 10/520,230

(22) PCT Filed: Jul. 9, 2003
(86) PCT No.: PCT/US03/21701

§ 371 Date: Feb. 2, 2005

Related U.S. Application Data
(60) Provisional application No. 60/399,056, filed on Jul. 26, 2002.

(51) Int. Cl. G01N 33/567

(52) U.S. Cl. 435/7.2

(57) ABSTRACT

Ligands, such as antibodies, are screened for binding to cell surface moieties such as receptors. Multiple cell types and/or binding ligands can be screened simultaneously in multiplexed assays. The screening assays employ membrane-anchored electrophoretic probes for detection of binding of the ligand to the cell surface.

### FIELD OF THE INVENTION

The present invention relates to methods and compositions for screening cell binding molecules, such as antibodies, preferably in multiplexed assays, such that multiple cell types and/or binding molecules can be screened simultaneously. The invention also relates to methods of labeling cell membranes and other membranes such that binding to such a membrane can be detected.

### BACKGROUND OF THE INVENTION

Determination of the binding behavior of cell membrane receptor proteins toward natural or artificial ligands is important for many biological and medical studies. In the field of target or drug discovery, high throughput screening efforts are key to isolating target-specific binders, agonists, or antagonists. Many of these therapeutic or diagnostic targets are cell surface antigens that, upon recognition by natural or synthetic binding molecules, trigger a network of signal transduction and gene regulation events inside the cell that result in cellular responses important in the initiation or maintenance of a disease. Target antigens may also differentially reside on the surface of cells and signify a unique state of physiology or disease progress in the tissue or organ. In researching these cell surface targets, the isolation of target-specific binders provides invaluable tools for detection and perturbation at the molecular level.

Monoclonal antibodies directed against tumor-associated antigens expressed on the tumor cell surface have found application in the immunodiagnosis and immunotherapy of human tumors. The interaction of certain monoclonal antibodies directed against tumor cells with cell surface antigens is well documented. See, e.g., H. Koprowski et al., Proc. Natl. Acad. Sci. USA 81:216-222 (1983); M. Herlyn et al., Adv. Cancer Res. 49:189-221 (1987); H. Koprowski et al., Somat. Cell. Mol. Genet., 11:297-302 (1985); H. F. Sears et al., Contr. Oncol. 19:180-192 (1984); H. Ross et al., Proc. Natl. Acad. Sci. USA 81:6681-6685 (1984); P. M. Grob et al., J. Biol. Chem. 260:8044-8049 (1985); and U. Murthy et al., Arch. Biochem. Biophys. 252:549-560 (1987).

Types of screening for cell binding moieties are very diverse. They include screening for monoclonal antibodies against biomarkers and therapeutic targets specific for diseased cells but not found on normal cells, and screening for ligands or pharmacological agents specific to surface antigens that are endogenously expressed or over-expressed as recombinant proteins on different cell lines.

Currently used screening strategies typically include a primary screen, which selects “hits” based on a desirable property, and one or more secondary screens, which determine the specificity of the selected property to the target in comparison to other related or irrelevant targets. Such primary and secondary screens can be time-consuming and are done sequentially, and additional development is required for each secondary assay. False positives and negatives in the primary screens can lead to unnecessary resource drain in the separate secondary screens. In the most widely employed current method of screening, cell types are screened individually, using a fluorescently labeled antibody, and the presence or absence of the antibody is determined, in a separate run for each cell type, by FACS sorting.

Other assays for receptor-ligand activity include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under Static Conditions 7.28.1-7.28.22); Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995. In these prior art assays, a number of different functional assays must be carried out on a number of different cell types to determine the binding affinity of an MAB or other ligand.

It would thus be desirable to combine primary and secondary screens in multiplexed screening methods that can simultaneously determine the reactivity of “hits” to specific targets and non-reactivity to others.

Currently employed receptor binding assays are increasingly difficult to carry out as the number of receptor molecules in the cell membrane decreases. Many receptors are normally expressed on the cell surface to only a small extent, i.e. a few hundred to a few thousand receptors/cell. These include many receptors types of great medical interest, including, for example, receptors for granulocyte/macrophage colony-stimulating factor (GM-CSF), many interleukins, erythropoietin, and tumor necrosis factor (TNF). Accordingly, screening methods for receptor binding which are highly sensitive and quantitative are also sought.

### SUMMARY OF THE INVENTION

The methods of the invention employ, in common, the use of membrane-anchored electrophoretic probes for detection of binding of a ligand to a cell surface. In one aspect, the invention provides a method of determining the binding specificity of a ligand to a cell surface moiety, the method comprising the steps of:

(a) providing one or more cell types such that each cell type has a different membrane-anchored electrophoretic probe, each membrane-anchored electrophoretic probe having an anchor moiety connected by a cleavable linkage to an electrophoretic tag, the tag having distinct optical or electrophoretic properties with respect to electrophoretic tags of other cell types;

(b) combining the one or more cell types with a ligand, and

(c) exposing the cell types and ligand to conditions such that at least one cleavable linkage is cleaved in at least one membrane-anchored electrophoretic probe of cells having a cell surface moiety to which said ligand is bound, whereby one or more electrophoretic tags are released; and

(d) electrophoretically separating and identifying the one or more released electrophoretic tags, to determine the specificity of the ligand for the cell surface moieties of the cell types.

The ligand, or cell binding moiety, may be, for example, an antibody, a secreted protein, and a small molecule candidate receptor agonist or antagonist. An embodiment of this method is illustrated in FIG. 1A.

In one embodiment, the exposing of step (c) comprises (i) attaching a proximity-dependent cleavage inducing group to each cell having a bound ligand, such that the membrane-anchored probes are cleavable only by such a cleavage inducing group which is on the same cell surface as the probe, and (ii) activating the cleavage inducing group. For example, the probes may be cleavable by a short-lived chemical species generated by a sensitizer group, such as a photosensitizer. In one embodiment, the sensitizer group is attached to the ligand; alternatively, the sensitizer group can be attached to a secondary molecule which forms a complex with the ligand. For example, when the ligand is an antibody, the sensitizer may be conjugated to a secondary antibody immunospecific against the antibody. In this case, the exposing of step (c) may comprise adding the secondary antibody and conjugated sensitizer to the cells and bound antibody.

In a further embodiment, the combining of step (b) further comprises isolating cell types having at least one ligand bound to at least one cell surface moiety. An embodiment of this method is illustrated in FIG. 1B. In this embodiment, the cleavable linkage may be, for example, chemically cleavable, photochemically cleavable, or enzymatically cleavable.

In a related aspect, the invention provides a method of identifying a cell surface antigen specific to substantially only one of a plurality of cell types, the method comprising the steps of:

(a) providing a plurality of cell types with cell surface antigens such that each cell type has a different membrane-anchored electrophoretic probe, the membrane-anchored electrophoretic probe having an anchor moiety connected by a cleavable linkage to an electrophoretic tag, said tag having distinct optical or electrophoretic properties with respect to electrophoretic tags of other cell types of the plurality;

(b) combining the one or more cell types with a candidate antibody,

(c) exposing the cell types and antibody to conditions such that at least one cleavable linkage is cleaved in at least one membrane-anchored electrophoretic probe of cells having a cell surface antigen to which said antibody is bound, whereby one or more electrophoretic tags are released;

(d) electrophoretically separating and determining the relative quantities of the one or more released electrophoretic tags to determine whether the candidate antibody binds to a cell surface antigen present on substantially only one of the plurality of cell types; and

(e) repeating steps (b)-(d) until a cell surface antigen specific to substantially only one of the plurality of cell types is identified.

In one embodiment, the method includes the further step of identifying the candidate antibody which binds to the cell surface antigen.

Again, the exposing of step (c) may comprise (i) attaching to each cell having a bound antibody, a proximity-dependent cleavage inducing group, such that each probe is cleavable only by such a cleavage inducing group on the same cell surface as the probe, and (ii) activating the cleavage inducing group. The cleavage-inducing group may be a sensitizer group which generates a short-lived chemical species, e.g. a photosensitizer. The group may be attached to the antibody; alternatively, the group may be conjugated to a secondary antibody immunospecific against the antibody. In this case, the exposing of step (c) includes adding the secondary antibody and conjugated sensitizer to the cells and bound antibody.

In a further embodiment, the combining of step (b) further comprises isolating cell types having at least one ligand bound to at least one cell surface moiety. In this embodiment, the cleavable linkage may be, for example, chemically cleavable, photochemically cleavable, or enzymatically cleavable.

Preferably, the relative quantities in step (d) are determined by measuring the area of tag peaks in an electropherogram of the released tags. A cell surface antigen specific to substantially only one of the plurality of cell types may be defined as one whose corresponding test tag peak in the electropherogram has an area that is at least 90%, preferably at least 95%, of the sum of the areas of all the test tag peaks in the electropherogram. Alternatively, it may be defined as one whose corresponding test tag peak in the electropherogram has an area that is at least twice, preferably at least four times, the area of the next largest test tag peak in the electropherogram.

In a further related aspect, the invention provides a further method of determining the binding affinity of a compound for a cell surface antigen. An embodiment of this method is illustrated in FIG. 2. In this method, one provides (a) one or more test cell-antibody pairs, each such pair comprising (i) a test cell having a membrane-anchored electrophoretic probe, the membrane-anchored electrophoretic probe having an anchor moiety connected by a cleavable linkage to an electrophoretic tag, the tag having distinct optical or electrophoretic properties with respect to electrophoretic tags of other test cells of the plurality, and (ii) at least one antibody specific for an cell surface antigen of the test cell of such pair, such cell surface antigen being different from cell surface antigens on other test cells of the plurality. The test compound is then (b) combined with the test cell-antibody pairs under conditions that permit the binding of the antibodies and the compound to one or more cell surface antigens recognized by the compound or antibodies; followed by (c) exposing the test cell-antibody pairs to conditions, as above, such that at least one cleavable linkage is cleaved in at least one membrane-anchored electrophoretic probe of cells having a cell surface antigen to which said antibody is bound, whereby one or more electrophoretic tags are released; (d) electrophoretically separating the released tags; and (e) determining the relative quantities of each of the one or more released electrophoretic tags to determine the binding affinity of the compound for the cell surface antigens.

In one embodiment, the method includes the further step of comparing the relative quantifies obtained in step (e) with those obtained when steps (a), (c) and (d) are carried out in the absence of step (b); that is, cleavage is carried out in the absence of the test compound.

The invention also provides a method of determining the binding specificity of a compound for an internalizing cell surface receptor. In this method, there is provided

(a) a plurality of test cell-antibody pairs, each such pair comprising (i) a test cell having a membrane-anchored electrophoretic probe, the membrane-anchored electrophoretic probe having an anchor moiety connected by a cleavable linkage to an electrophoretic tag having distinct optical or electrophoretic properties with respect to electrophoretic tags of other test cells of the plurality, and (ii) at least one antibody effective to bind to an internalizing cell surface receptor of the test cell. Preferably, the receptor of each cell is different from internalizing cell surface receptors on other test cells of the plurality.

The plurality of test cell-antibody pairs is then combined with (b) the compound, under conditions that permit the endocytosis of complexes that form between the compound and one or more of the internalizing cell surface receptors, followed by (c) the plurality of antibodies, under conditions that permit binding to internalizing cell surface receptors. The plurality is then (d) exposed to conditions, as above, such that at least one cleavable linkage is cleaved in at least one membrane-anchored electrophoretic probe of cells having an internalizing cell surface receptor to which said antibody is bound, whereby one or more electrophoretic tags are released. The released tags are electrophoretically separated, and the relative quantities of each of the one or more released electrophoretic tags are determined, to determine the specificity of the compound for the plurality of endocytosing cell surface receptors.

In one embodiment, the method further comprises the step of comparing the relative quantities obtained in step (f) with those obtained when steps (a) and (c)-(e) are carried out in the absence of step (b); that is, the procedure is carried out in the absence of added test compound.

These and other objects and features of the invention will become more fully apparent when the following detailed description of the invention is read in conjunction with the accompanying drawings.

### BRIEF DESCRIPTION OF THE DRAWINGS

FIG. 1A illustrate an embodiment of the invention for screening a ligand for its ability to bind to cell-surface moieties present in one or more of a plurality of different cell types (multiplex screening), where the cells are labeled with surface bound cleavable electrophoretic probes;

FIG. 1B illustrates an embodiment of the invention as described for FIG. 1A, carried out in a heterogeneous format;

FIG. 2 illustrates an embodiment of the invention for determining the binding affinity of a test compound for a cell surface moiety, in a competitive assay format;

FIG. 3 shows the correlation of signal from released eTag with the concentration of eTag incubated with a HL-60 cell sample;

FIGS. 4A-B illustrate the detection of primary antibodies bound to eTag-labeled cell surfaces, showing the correlation of signal with level of antibody;

FIGS. 5-6 illustrate the detection of primary antibodies bound to subpopulations of eTag-labeled cells in mixed cell assays;

FIGS. 7A-K shows several representative lipophilic groups;

FIGS. 8A-B shows cleavable linkages based on thiazole and oxazole groups, respectively, and their cleavage products, where G is a membrane anchoring group and the open valence represents a link to an electrophoretic tag moiety;

FIG. 8C shows a cleavable linkage based on a heteroatom-substituted olefin and its cleavage by singlet oxygen;

FIG. 9 shows a thioether cleavable linkage and its cleavage by singlet oxygen;

FIGS. 10A-C show steps in the attachment of an eTag, via a thioether cleavable linkage, to a group bearing a thiol functionality;

FIGS. 11A-C illustrate exemplary methods for attaching a membrane anchoring group to an eTag;

FIGS. 12A-B illustrate exemplary methods for attaching a sensitizer to a biotin derivative; and

FIGS. 13A-C illustrate steps in practicing the methods of the invention using a microfluidics/CE device.

### DETAILED DESCRIPTION OF THE INVENTION

I. Definitions

Unless otherwise defined below, the terms used herein have their normally accepted scientific meanings. Definition of standard chemistry terms may be found in reference works, including Carey and Sundberg (1992) “Advanced Organic Chemistry 3^{rd} Ed.”, Vols. A and B, Plenum Press, New York. The practice of the present invention will employ, unless otherwise indicated, conventional methods of mass spectroscopy, protein chemistry, biochemistry, recombinant DNA techniques and pharmacology, within the skill of the art. Such techniques are explained fully in the literature. See, for example, G. Barany and R. B. Merrifield (1980), “The Peptides: Analysis, Synthesis, Biology”, Vol. 2, E. Gross and J. Meienhoffer, eds., Academic Press, New York.; Methods In Enzymology (S. Colowick and N. Kaplan, eds., Academic Press, Inc.); Remington's Pharmaceutical Sciences, 18th Edition (Easton, Pa.: Mack Publishing Company, 1990).

All publications, patents and patent applications cited herein, whether supra or infra, are hereby incorporated by reference in their entirety.

As used in this specification and the appended claims, the singular forms “a”, “an” and “the” include plural referents unless the content clearly dictates otherwise. Thus, for example, reference to “an oligonucleotide” includes a mixture of two or more oligonucleotides, and the like.

In describing the present invention, the following terms will be employed, and are intended to be defined as indicated below.

A “membrane anchored electrophoretic probe” refers to a chemical structure containing (1) an electrophoretic “tag”, (2) a cleavable linkage and (3) a lipophilic anchor moiety. Sets of probes are generally provided for use in the methods described herein. Cleavage of the probe at the cleavable linkage releases the electrophoretic tag (also referred to as an eTag, eTag reporter, or eTag marker) which contains a detectable label and a mobility modifying group. Such probes are described in detail in Section III.

“Electrophoretic mobility” refers to the mobility of a charged compound through a defined separation medium, and under defined buffer and electric field conditions. “Different electrophoretic mobilities,” as applied to tags, means that the tags are separable from one another on the basis of different rates of migration in a given electrophoretic medium, e.g., acrylamide gel, and under defined electrophoretic conditions, e.g., standard electrophoretic conditions for separating either positively or negatively charged compounds with different charge/mass ratios.

“Electrophoretic resolution” is a measure of the distinctness, or lack of overlap, of adjacent peaks in an electropherogram. It can be defined, for example, as the distance between adjacent peak maximums divided by four times the larger of the two standard deviations of the peaks. Preferably, adjacent peaks have a resolution of at least 1.0, and more preferably, at least 1.5, and most preferably, at least 2.0. In a given separation and detection system, the desired resolution may be obtained by selecting a plurality of electrophoretic tags whose members have electrophoretic mobilities that differ by at least a peak-resolving amount, such quantity depending on several factors well known to those of ordinary skill, including signal detection system, nature of the fluorescent moieties, the diffusion coefficients of the tags, the presence or absence of sieving matrices, nature of the electrophoretic apparatus, e.g. presence or absence of channels, length of separation channels, and the like.

The term “alkyl” as used herein refers to a straight, branched, or cyclic hydrocarbon chain fragment or radical containing between about one and about fifty carbon atoms, more preferably between about one and about twenty carbon atoms (e.g., methyl, ethyl, n-propyl, iso-propyl, cyclopropyl, n-butyl, iso-butyl, tert-butyl, cyclobutyl, adamantyl, noradamantyl and the like). Straight, branched, or cyclic hydrocarbon chains having eight or fewer carbon atoms will also be referred to herein as “lower alkyl”. The hydrocarbon chains may further include one or more degrees of unsaturation, i.e., one or more double or triple bonds (e.g., vinyl, propargyl, allyl, 2-buten-1-yl, 2-cyclopenten-1-yl, 1,3-cyclohexadien-1-yl, 3-cyclohexen-1-yl and the like). Alkyl groups containing double bonds such as just described are also referred to herein as “alkenes”. Similarly, alkyl groups having triple bonds are also referred to herein as “alkynes”. However, as used in context with respect to cyclic alkyl groups, the combinations of double and/or triple bonds do not include those bonding arrangements that render the cyclic hydrocarbon chain aromatic.

Representative examples of alkanes useful as alkyl group substituents of the present invention include methane, ethane, straight-chain, branched or cyclic isomers of propane, butane, pentane, hexane, heptane, octane, nonane and decane, with methane, ethane and propane being preferred. Alkyl groups having up to about thirty, or up to about fifty carbon atoms are contemplated in the present invention.

Representative examples of alkenes useful as alkenyl group substituents include ethene, 1-propene, 2-propene, and straight-chain, branched or cyclic isomers of butene, pentene, hexene, heptene, octene, nonene and decene, with ethene and propene being preferred. Alkenyl groups having up to about thirty or fifty carbon atoms, and up to about five double bonds, or more preferably, up to about three double bonds are contemplated in the present invention.

Representative examples of alkynes useful as alkynyl group substituents include ethyne, propyne, straight-chain or branched isomers of butyne, pentyne, hexyne, heptyne, and straight-chain, branched, or cyclic isomers of octyne, nonyne and decyne, with ethyne and propyne being preferred. Alkynyl groups having up to about thirty, or up to about fifty carbon atoms, and having up to about five or up to about three triple bonds are contemplated in the present invention.

The term “halo” or “halogen” as used herein refers to the substituents fluoro, bromo, chloro, and iodo.

The term “carbonyl” as used herein refers to the functional group —C(O)—. It will be appreciated that this group can frequently be replaced with well-known carbonyl equivalent groups having similar electronic and/or steric character, such as thiocarbonyl (—C(S)—); sulfinyl (—S(O)—); sulfonyl (—SO₂—); phosphonyl (—PO₂—), and methine. Other carbonyl equivalents will be familiar to those having skill in organic chemistry.

The term “aryl” as used herein refers to cyclic aromatic hydrocarbon chains having twenty or fewer carbon atoms, e.g., phenyl, naphthyl, phenanthryl, biphenyl and anthryl. One or more carbon atoms of the aryl group may also be substituted with, e.g.: alkyl; aryl; heterocycle; halogen; nitro; cyano; carboxy; hydroxyl, alkoxyl or aryloxyl; thio or mercapto; alkyl- or arylthio; amino; alkylamino; arylamino; dialkyl-, diaryl-, or arylalkylamino; aminocarbonyl; alkylaminocarbonyl; arylaminocarbonyl; dialkylaminocarbonyl; diarylaminocarbonyl or arylalkylaminocarbonyl; carboxyl; alkyl- or aryloxycarbonyl; carboxaldehyde; aryl- or alkylcarbonyl; iminyl; aryl- or alkyliminyl; sulfo; alkyl- or arylsulfonyl; hydroximinyl; aryl- or alkoximinyl; carbamido; or thiocarbamido. In addition, two or more alkyl or heteroalkyl substituents of an aryl group may be combined to form fused aryl-alkyl or aryl-heteroalkyl ring systems (e.g., tetrahydronaphthyl). Substituents including heterocyclic groups (e.g., heterocycloxy, heteroaryloxy, and heteroaralkylthio) are defined by analogy to the above-described terms.

The term “aralkyl” as used herein refers to an aryl group that is joined to a parent structure by an alkyl group as described above, e.g., benzyl, α-methylbenzyl, phenethyl, and the like.

The term “heterocycle” as used herein refers to a cyclic alkyl group or aryl group as defined above in which one or more carbon atoms have been replaced by a non-carbon atom, especially nitrogen, oxygen, or sulfur. Non-aromatic heterocycles will also be referred to herein as “cyclic heteroalkyl”. Aromatic heterocycles are also referred to herein as “heteroaryl”. For example, such groups include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridazinyl, triazinyl, piperidinyl, morpholinyl, thiomorpholinyl, pyrazinyl, piperazinyl, pyrimidinyl, naphthyridinyl, benzofuranyl, benzothienyl, indolyl, indolinyl, indolizinyl, indazolyl, quinolizinyl, quinolinyl, isoquinolinyl, cinnolinyl, phthalazinyl, quinazolinyl, quinoxalinyl, pteridinyl, quinuclidinyl, carbazolyl, acridiniyl, phenazinyl, phenothiazinyl, phenoxazinyl, purinyl, benzimidazolyl, benzthiazolyl, and benzoxazolyl.

A “protein” or a “polypeptide” is used in its broadest sense to refer to a compound of two or more subunit amino acids, amino acid analogs, or other peptidomimetics. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein. Full-length proteins, analogs, and fragments thereof are encompassed by the definition. The terms also include postexpression modifications of the polypeptide, for example, glycosylation, acetylation, phosphorylation and the like. Furthermore, as ionizable amino and carboxyl groups are present in the molecule, a particular polypeptide may be obtained as an acidic or basic salt, or in neutral form. A polypeptide may be obtained directly from the source organism, or may be recombinantly or synthetically produced.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens) and the like. The term “fluorescer” refers to a substance or a portion thereof which is capable of exhibiting fluorescence in the detectable range.

As used herein, a “solid support” refers to a solid surface such as a magnetic bead, latex bead, microtiter plate well, glass plate, nylon, agarose, acrylamide, and the like.

A “liposome” refers to a generally spherical cluster or aggregate of amphiphilic compounds, typically in the form of one or more concentric layers, for example, bilayers. The liposomes may be formulated, for example, from phosphorus or silicon amphiphilic or conventional amphiphilic compounds, such as lipids, including ionic and/or non-ionic lipids, and/or a combination phosphorus and/or silicon amphiphilic compounds and conventional amphiphilic compounds.

A “micelle” refers to a colloidal entity formulated from amphiphilic compounds, including the phosphorus or silicon amphiphilic compounds, as well as conventional lipids. Micelles may comprise a monolayer, hexagonal H2 phase configuration or a bilayer configuration.

“Antibody” refers to an immunoglobulin that specifically binds to, and is thereby defined as complementary with, a particular spatial and polar organization of another molecule. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained.

A “monoclonal antibody” MAB) is an immunoglobulin produced by a single clone of lymphocytes, i.e. the progeny of a single B cell, which recognizes only a single epitope on an antigen. Antibodies are discussed further in Section VI below.

An “antibody binding composition” is a molecule or complex of molecules that comprises one or more antibodies and derives its binding specificity from an antibody. Antibody binding compositions include, but are not limited to, antibody pairs in which a first antibody binds specifically to a target molecule and a second antibody binds specifically to a constant region of the first antibody; a biotinylated antibody that binds specifically to a target molecule and to streptavidin derivatized with moieties such as electrophoretic tags or photosensitizers; antibodies specific for a target molecule and conjugated to a polymer, such as dextran, which, in turn, is derivatized with moieties such as electrophoretic tags or photosensitizers; and antibodies specific for a target molecule and conjugated to a bead, or microbead, or other solid phase support, which, in turn, is derivatized with moieties such as electrophoretic tags or photosensitizers, or polymers containing the latter.

“Capillary electrophoresis” refers to electrophoresis in a capillary tube or in a capillary plate, where the diameter of the separation column or thickness of the separation plate is between about 25-500 microns, allowing efficient heat dissipation throughout the separation medium, with consequently low thermal convection within the medium.

A “sieving matrix” or “sieving medium” refers to an electrophoresis medium that contains crosslinked or non-crosslinked polymers which are effective to retard electrophoretic migration of charged species through the matrix.

As used herein, the term “spectrally resolvable”, in reference to a plurality of fluorescent labels, means that the fluorescent emission bands of the labels are sufficiently distinct, i.e. sufficiently non-overlapping, that electrophoretic tags to which the respective labels are attached can be distinguished on the basis of the fluorescent signal generated by the respective labels using standard photodetection systems, e.g. employing a system of band pass filters and photomultiplier tubes, or the like, as exemplified by the systems described in U.S. Pat. Nos. 4,230,558, 4,811,218, or the like, or in Wheeless et al., in Flow Cytometry: Instrumentation and Data Analysis (Academic Press, New York, 1985), pp. 21-76.

“Specific”, in reference to the binding of two molecules or a molecule and a complex of molecules, refers to the specific recognition of one for the other and the formation of a stable complex, as compared to substantially less recognition of other molecules and the lack of formation of stable complexes with such other molecules. Preferably, “specific”, in reference to binding, means that to the extent that a molecule forms complexes with other molecules or complexes, it forms at least fifty percent of the complexes with the molecule or complex for which it has specificity. Generally, the molecules or complexes have areas on their surfaces or in cavities giving rise to specific recognition between the two binding moieties. Exemplary of specific binding are antibody-antigen interactions, enzyme-substrate interactions, polynucleotide hybridizations and/or formation of duplexes, cellular receptor-ligand interactions, and so forth.

A “multiplexed assay” refers to an assay in which multiple assay reactions are carried out in a single reaction chamber and/or and analyzed in a single separation and detection format.

II. Assays of the Invention

Before describing the present invention in detail, it is to be understood that the invention is not limited to particular formulations or process parameters, as such may, of course, vary. It is also to be understood that the terminology used herein is for the purpose of describing particular embodiments of the invention only, and is not intended to be limiting.

The present invention provides, in one aspect, compositions and methods for labeling membranes, such as cellular membranes and liposomes, such that the label can be detected. The present methods are practiced by labeling the membranes with these compounds, which may be referred to as “electrophoretic probes”, and comprise the formula G-(L-E)ₘ. As described in detail in Section III below, the compounds comprise a lipophilic group (G), a cleavable linkage (L), and one or more electrophoretic groups (E), where m can be between 1 and 1000, preferably between 1 and 100, and more preferably between 1 and 10. In compounds having more than one electrophoretic group, the groups E can be the same or can be different, such that each E is capable of being individually detected. By cleaving linkage L on probes bound to cells, electrophoretic tags E are released and can be detected.

The methods of the invention can be used to determine the binding ability and/or specificity of a test compound, or a group of test compounds, to a cell surface moiety, such as a cell surface receptor. The assays typically employ a plurality of different target cells. Each different type of cell is provided with a distinct electrophoretic probe, bound to the cell surface via a cleavable linker. In general, binding of a test compound to a cell is effective to induce cleavage of the linker and release of a characteristic “tag” portion of the electrophoretic probe. By detecting the type and amounts of released “tags”, information about binding of different compounds to different cell types can be obtained.

The test compound, or test ligand, may be, for example, an antibody, particularly a monoclonal antibody, a peptide ligand, such as a secreted peptide, a pharmacological agent, or a candidate pharmacological agent. Antibodies are discussed in more detail below. Candidate pharmacological agents include biomolecules as well as small molecule compounds, which may be organometallic, inorganic, or, more typically, organic compounds, which are screened for binding to a cell surface moiety such as a receptor. Receptor binding compounds may be agonists (which produce a physiologic reaction similar to that of a naturally occurring receptor binding ligand) or antagonists (which interfere with the physiological action of the receptor).

The different cell types may include, for example, cells of similar disease category (e.g. ovarian cancers, breast cancers), so that the identified cell surface marker can be confirmed with confidence of its expression consistency with the disease. The multiplex samples may also include cells of normal and irrelevant disease phenotypes from the same tissue origin, so that the disease specificity of the identified cell surface marker can be determined. A typical multiplex assay reaction may contain 5-10 different cell samples. In a simple duplex assay reaction, only normal and diseased cells are included. (Any of the assays described herein could also be carried out with one type of cell, to determine the binding affinity of a compound or compounds for that cell type, but are used to greatest advantage in the multiplex format illustrated.)

A. Homogeneous Assay Format

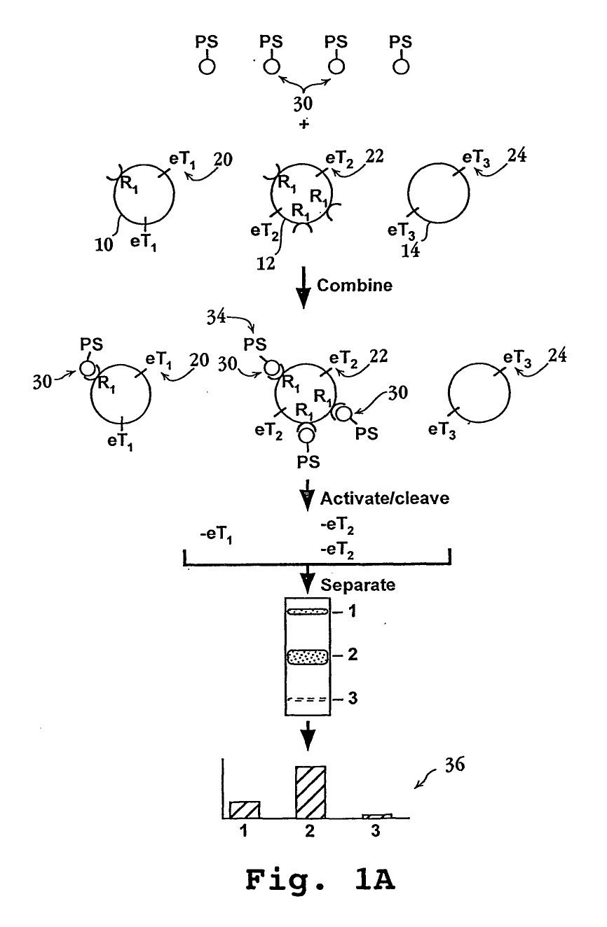

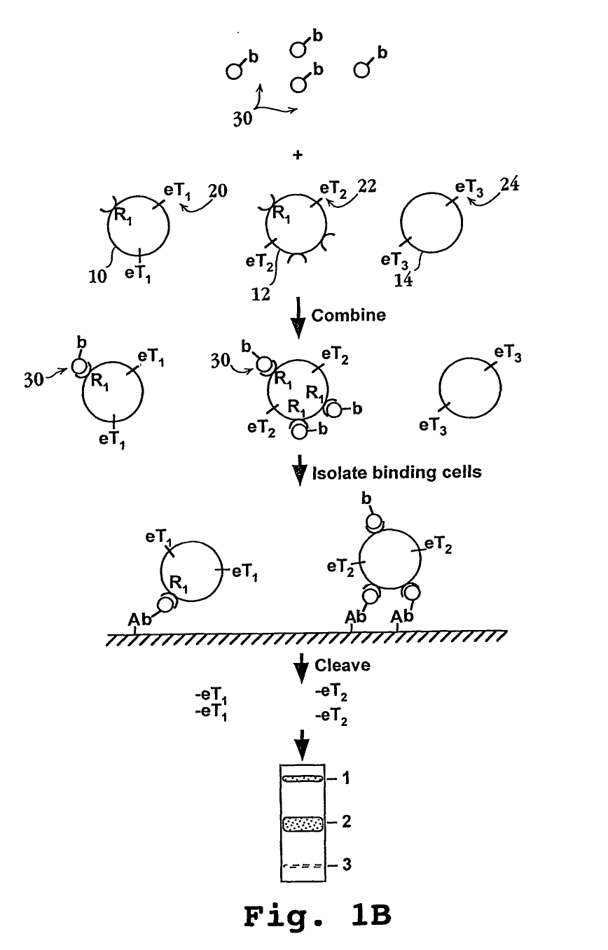

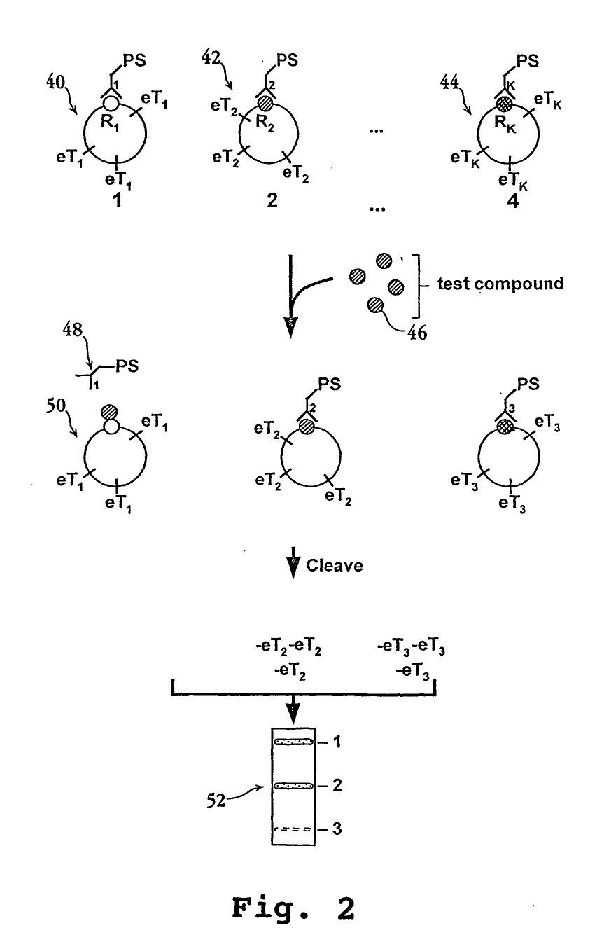

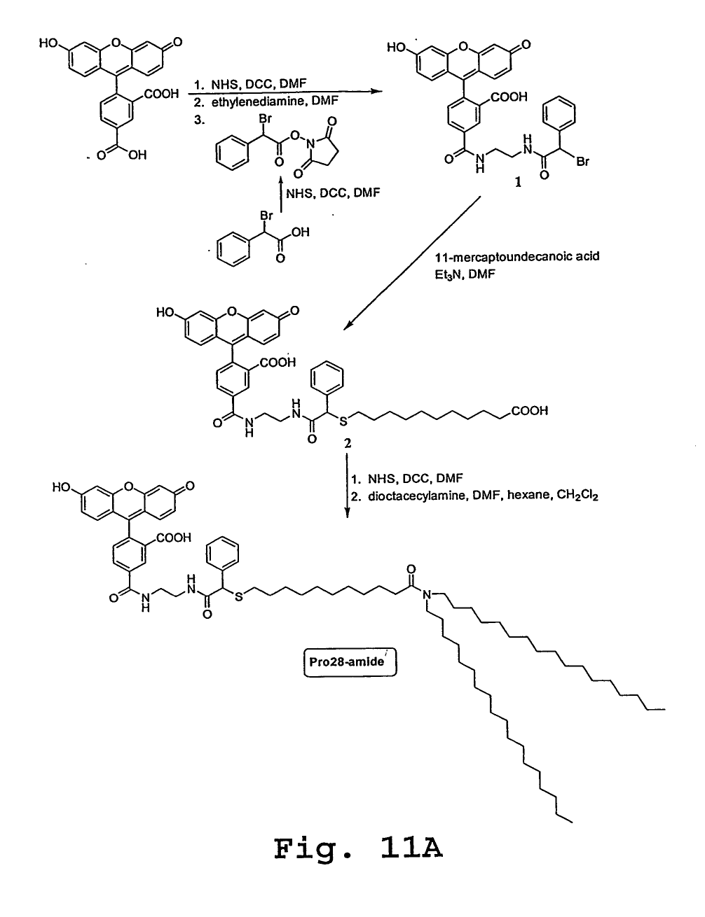

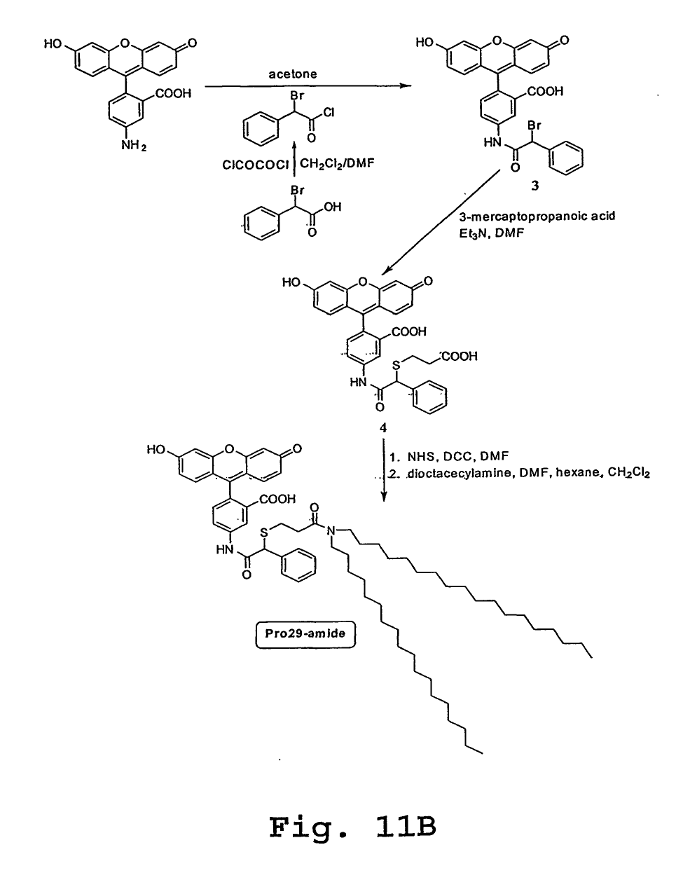

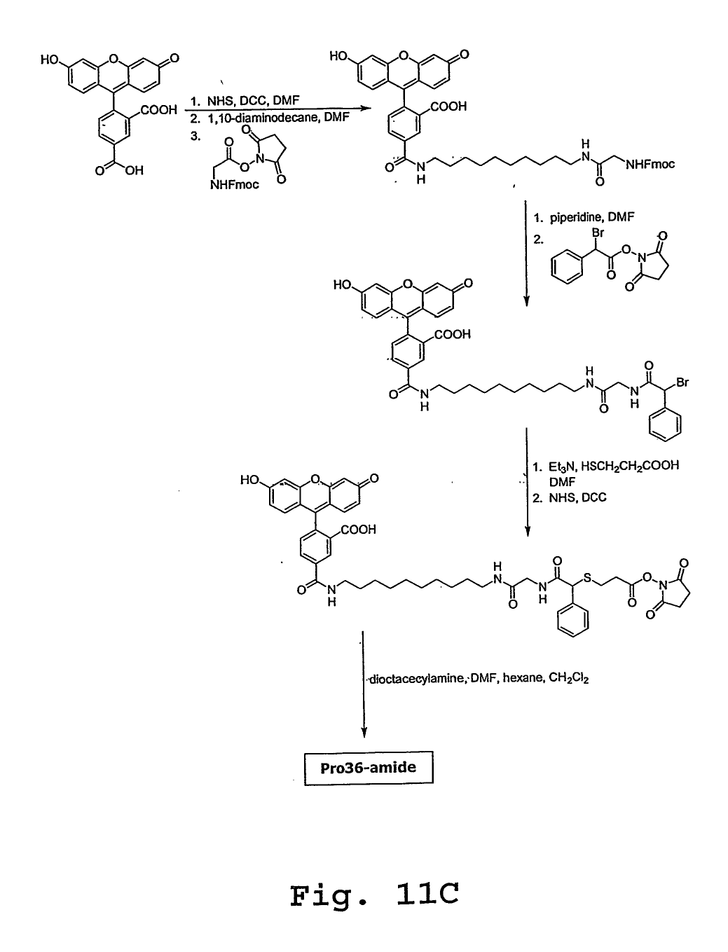

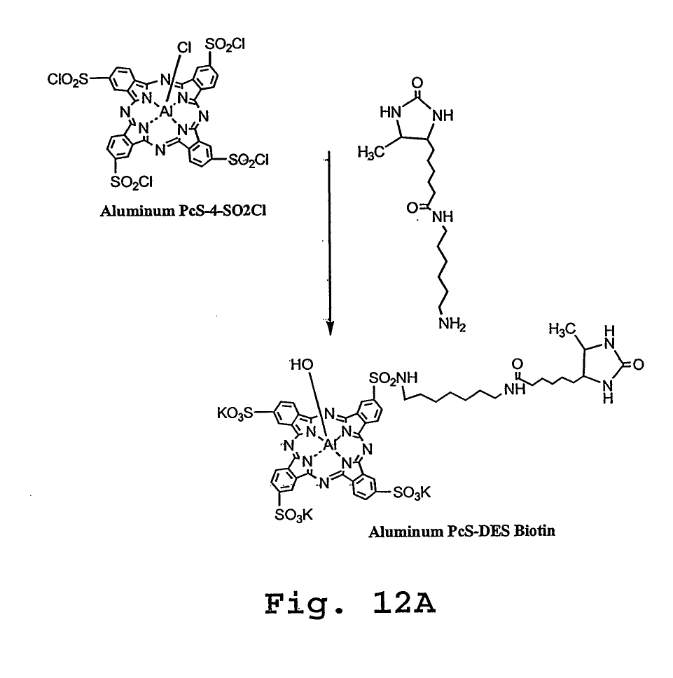

FIG. 1A illustrates an exemplary method for determining the cell surface binding affinity of a test compound among a plurality of cell types. FIG. 1A shows a plurality of cells 10, 12, 14 of different cell types. Each cell may or may not contain a surface moiety recognized by the test compound, such as receptor R1 in the Figure. Each cell type is labeled with a cell-specific electrophoretic probe 20, 22, 24 (also designated eT₁, eT₂, eT₃), which is anchored to the cell via a cleavably linked anchor moiety, as discussed further below. Preferably, the cleavably linked anchor moiety is lipophilic (see e.g. FIGS. 11A-C), e.g. an alkyl chain about 16-18 carbons in length, which is inserted into the plasma membrane. Other methods of anchoring are also available; for example, the probe may include a cleavably linked biotin, in which case the probe is conjugated to streptavidin, which binds to biotinylated lipids, e.g. phospholipids, which are inserted into the plasma membrane.

The lipophilic anchor moieties of the probes can be incorporated into the cell membrane by incubation for about an hour at 37° C./5% CO₂. Preferably, the cells are then chilled, e.g. 4° C., for the binding assay. At these low temperatures, leaching of incorporated lipophilic probes from the cell surface into the extracellular environment is minimized, and cross-labeling of different cell populations with different probes is avoided.

With continued reference to FIG. 1A, the mixture of cells is incubated, e.g. at 4° C. for one hour, with a test compound 30. In the embodiment of FIG. 1A, where the assay is carried out in a homogeneous format, each test compound is conjugated, directly or indirectly, to a cleavage-inducing group.

The cleavage-inducing group may be linked directly to the test compound, or it may be linked to a secondary compound which binds to the test compound, e.g. a secondary antibody effective to bind to any test MAB. Alternatively, the test compound, or the secondary antibody, may be labeled with biotin, in which case the mixture can be incubated with streptavidin-conjugated photosensitizer. (See Examples 5-8, below.)

The cleavage-inducing group is effective to cleave the linkages of electrophoretic probes on cells only within a limited proximity, effectively on the same cell as the bound compound. An example is a sensitizer group (indicated by 34) that is capable of generating a short-lived active chemical species, such as singlet oxygen, under selected activation conditions, e.g., photoillumination. Accordingly, activation of photosensitizer (PS) groups in such an assay mixture will cleave electrophoretic probes only if the probes are in close proximity to said photosensitizer group; that is, on the same cell surface to which a test compound is bound.

As can be appreciated from FIG. 1A, binding of test compound to a cell places the conjugated sensitizer group in proximity with the cleavable linker of the electrophoretic probe anchored to the cell surface. The cell mixture is then treated to activate the cleaving groups, i.e. illuminated to active photosensitizers (e.g. at 640-800 nm for a phthalocyanine sensitizer), generating singlet oxygen in the proximity of the bound sensitizer groups. The singlet oxygen is sufficiently short-lived that only surface-bound sensitizer, as opposed to any unbound, solution-phase sensitizer, is effective to cleave electrophoretic probes bound to cells. The reaction thus selectively cleaves the electrophoretic probes on cells to which the test compound is bound, such as probes 20 and 22 in FIG. 1A, releasing the specific “tags” from the probes labeling these cell types.

The released tags are then separated and analyzed, preferably by electrophoresis. The separated peaks are detected, e.g. by fluorescence emission detection of fluorescent labels in the tags. Because the separation characteristics of the eTags released from the respective cells are known, the multiplex data output, shown schematically at 36 in FIG. 1A, can be used to identify the cell samples to which the test compound binds. Intensity of peaks can also be used to determine the relative extent of binding of the test compound to different cells, if the test compound binds to more than one cell.

Preferably, the labels employed in the electrophoretic tags are such that peak height or area of different tags can be directly correlated to the number of tags detected. For example, a set of probes may employ the same label and different mobility modifying groups, as discussed further below.

A known amount of a “standard” eTag may be added to the test assay to provide a standard for calibrating the mobility and peak characteristics of the released tag(s). A positive control antibody (e.g. anti-HLA-A,B,C) can also be included, to ensure detection of release of different eTags from different cell samples. The measured peak height or area under the curve (AUC) of the standard eTag (T_{std}), relative to the known amount of standard eTag added, can be used to calculate the amount of test and control eTags from the measured peak heights or AUC in the electropherogram.

B. Heterogeneous Assay Formats

The assays may also be carried out in a heterogeneous format, as illustrated in FIG. 1B. Heterogeneous techniques normally involve a separation step, where cells having bound ligand are separated from other assay components, e.g. non-binding cells and unbound ligand. Homogeneous assays do not require, but may employ, a separation step. Separation can be achieved in a variety of ways, each employing a reagent bound to a solid support that distinguishes between binding and non-binding cells. The solid support may be a vessel wall, e.g., microtiter well plate well, capillary, plate, slide, beads, including magnetic beads, liposomes, or the like. The primary characteristics of the solid support are that it (1) permits segregation of the binding cells from non-binding cells and (2) does not interfere with the formation of the binding complex, nor the other operations of the determination.

The solid support may bind the cell-ligand complex directly or indirectly. For direct binding, the surface may be activated with various functionalities that will form covalent bonds with a test binding compound. For indirect binding, which is preferred, the surface noncovalently binds the test compound, or it binds an intermediate compound, such as biotin, which is linked to the test compound.

A heterogeneous assay is illustrated in FIG. 1B. After binding of the test compound to the differently labeled cell types, as above, the binding cells are separated from non-binding cells. This can be accomplished, for example, by employing test compounds linked to an affinity molecule, such as biotin, and capturing cells containing bound test compound on a surface containing a binding partner for the affinity molecule, such as streptavidin (as depicted in FIG. 1B).

The non-binding cells and/or unbound ligand are generally removed by washing the support. Where particles or beads are employed, these may be separated from the supernatant before washing, by filtration, centrifugation, magnetic separation, etc.

The captured cells are then treated, as above, to cleave the attached electrophoretic probes. In a this format, cleavage of the electrophoretic probes on binding cells need not be proximity dependent, since cells not having bound ligand have been removed. Therefore, a larger variety of cleavage protocols can be used. Cleavage may still employ a sensitizer, as described above, to cleave an oxidatively labile linkage, but it may also employ various types of chemical, photochemical, or enzymatic cleavage of a variety of cleavable linking groups, such as are known in the art. For example, non-limiting examples of chemically cleavable linkages include disulfides (cleavable by reduction, typically using dithiothreitol), azo groups (cleavable with dithionate), sulfones (cleavable with basic phosphate, with or without dithiothreitol), glycols, cleavable by periodate, and esters, cleavable by hydrolysis. Photolabile linkers include, for example, azo linkages and o-nitrobenzyl ethers.

After washing, the support may be combined with a solvent into which the e-tag reporters are to be released. Depending on the nature of the cleavable bond and the method of cleavage, the solvent may include any additional reagents for the cleavage. Where reagents for cleavage are not required, the solvent is conveniently an electrophoretic buffer. For example, where the cleavable linkage is photolabile, the medium may be irradiated with light of appropriate wavelength to release the e-tag reporters into the buffer.

If the cleavage reagent should interfere with electrophoretic analysis, it may be necessary to separate the e-tag reporters from the cleavage reagent solution. Depending on the nature of the e-tag reporters and the reagent, the e-tag reporters may be sequestered from the reagent by using ion exchange columns, liquid chromatography, an initial electrophoretic separation, and the like. Alternatively, a capture ligand can be bound to the e-tag moiety, to remove any interfering components in the mixture.

Following release of the electrophoretic tags from the probes, the tags are separated by electrophoresis and analyzed as above.

C. Assays for Specific Binding

In selected embodiments, the assays can be used to identify a cell surface moiety, such as a receptor or antigen, specific to substantially only one of the plurality of cell types employed in the assays. Candidate binding compounds, preferably antibodies, are exposed to the probe-labeled cells, such that probes are cleaved only from cells to which the candidate compound binds, in the manner described above. Released eTags for each candidate binding compound are separated by electrophoresis and identified. The assay is repeated with a series of candidate binding compounds, if necessary, until a cell surface moiety specific to substantially only one of the plurality of cell types is identified; that is, the candidate binding compound binds to substantially only one of the plurality of cell types.

The criteria for “substantially only one” can be based, for example, on relative peak heights or areas in an electropherogram of the cleaved tags produced for each binding compound. For example, where the area of one test eTag peak in an electropherogram is at least 90%, preferably at least 95%, more preferably at least 98%, and most preferably at least 99%, of the sum of the areas of all the test eTag peaks in an electropherogram of eTags released by binding of a test compound, the cell corresponding to that test eTag peak can be considered to have a cell surface moiety not present in the other cell types of the tested plurality. Other valid criteria could be employed; for example, where the area, or height, of one test eTag peak in an electropherogram is at least twice, preferably at least three times, and more preferably at least four times the area, or height, of the next largest peak in an electropherogram of eTags released by binding of a test compound, the cell corresponding to that test eTag peak can be considered to have a cell surface moiety not present in the other cell types of the tested plurality. This procedure can also be used to identify a binding compound, such as an antibody, that binds to a cell surface moiety specific to substantially only one of a plurality of cell types, using similar criteria.

D. Competitive Assay Formats

The assays can also be carried out in a competitive format, as shown in FIG. 2. In this embodiment, each of the plurality of cell types is paired not only with a particular electrophoretic probe, but also with a specific binding partner, typically an antibody. The antibody is linked, directly or indirectly, to a proximity-dependent (in a homogenous format) cleavage inducing moiety, such as a photosensitizer. A plurality of such cell-antibody binding pairs, each labeled with a specific electrophoretic probe as described above, is shown at 40, 42, 44 in FIG. 2.

A test compound 46 is then incubated with this mixture. If the test compound successfully competes for binding with antibody in any of the cell-antibody binding pairs, the bound antibody will be displaced, as shown at 48 in FIG. 2. (Alternatively, the antibodies may be added following, or concurrent with, addition of the test compound.) Following incubation, the assay mixture is treated to activate the cleavage-inducing moieties on the antibodies and thus cleave electrophoretic probes in the vicinity, that is, on cells to which antibodies are bound. Cells to which test compound binds, such as cell 50 in FIG. 2, will have less surface-bound antibody and thus less cleavage-inducing moiety available. Cleavage and tag release from these cells will be correspondingly reduced, and less of the corresponding tag will be detected in the cleavage mixture, as shown at 52.

Accordingly, the electrophoretic data can be used to determine to which cells the test compound binds. In this case, increased binding is indicated by a reduction in signal.

The competitive assay format is particularly useful for detection of binding to internalizing or “endocytosing” receptors; that is, receptors, such as G-protein coupled receptors (GPCR's), which are internalized into cells as a result of their activation by ligands. In this embodiment, a cell containing such receptors is labeled with a cleavable electrophoretic probe, as described above. Also provided is an antibody which is effective to bind to the receptor. (If necessary, the receptor may be expressed with an antibody-binding group.) The antibody is conjugated, directly or indirectly, with a proximity-dependent cleavage-inducing moiety, such as a photosensitizer.

Preferably, a plurality of such cell-antibody pairs is used. Each cell type of the plurality has a different internalizing receptor and binding antibody, and is labeled with a particular electrophoretic probe.

A test compound is then introduced to the plurality of cells, under conditions such that, if the compound binds to the receptor, the receptor internalizes. Following a suitable incubation period, the corresponding plurality of specific antibodies are introduced and allowed to bind to available receptors on the corresponding cell types. The cleavage inducing moieties on the antibodies (or on secondary molecules conjugated to the antibodies) are then activated, as above, to cleave the probes on the cells to which they are bound.

If the test compound binds to receptors on a particular cell, and the receptor internalizes, fewer targets are available for binding of the antibody, reducing the amount of photosensitizer which is able to bind to the cell surface. Thus, for cells to which test ligands bind, a decrease of signal from cleaved eTags is observed. Accordingly, the absence of an eTag peak in the electropherogram indicates strong binding of the test ligand to the internalizing receptor of the corresponding cell.

E. Examples

Examples 5-7 illustrate the quantitative nature of the assays. In the procedures described therein, cells were labeled with a lipophilic electrophoretic probe designated “Pro28” (see Example 1 and FIG. 11A) and washed to remove unincorporated probe. Labeled cells were then incubated with primary antibody, washed to remove unbound antibody, then incubated with biotinylated secondary antibody, which binds to the bound primary antibody on the cells, followed by streptavidin-photosensitizer beads, thus linking the photosensitizer to the cell surface. The electrophoretic tags (fluorescently labeled) were cleaved from the cell-bound probes by photoactivation, and the tag release mixture was analyzed by electrophoresis.

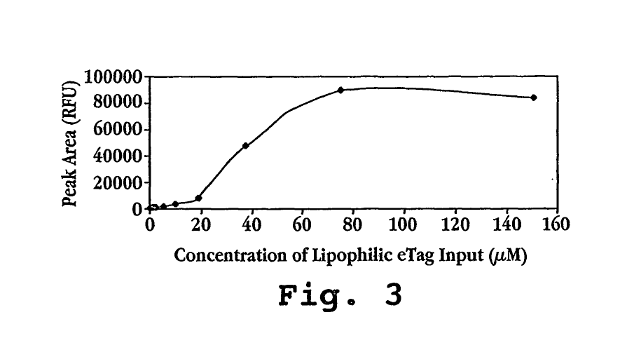

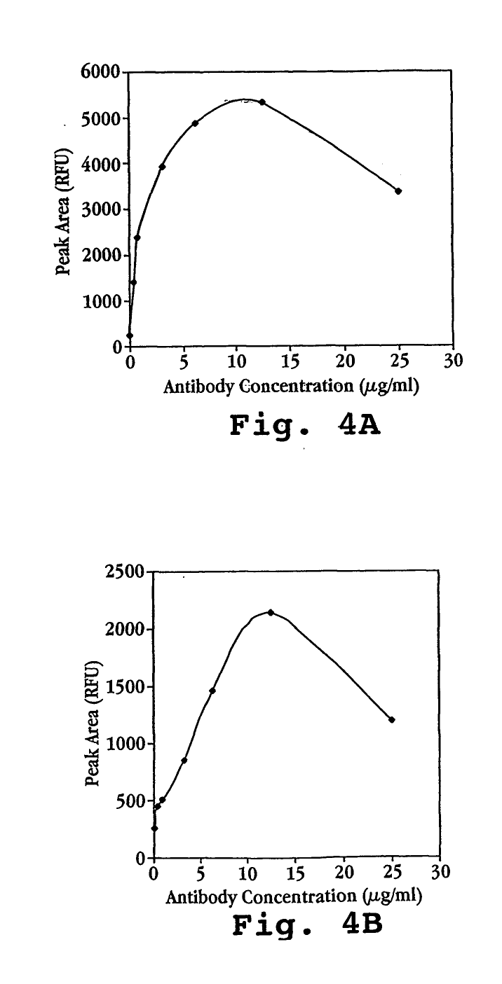

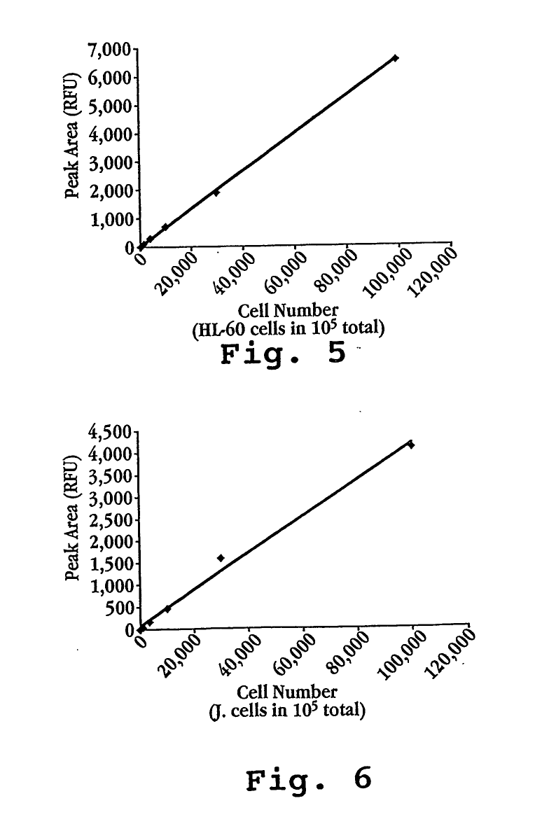

In Examples 5-6, varying amounts of probe and of primary antibody were employed, and the resulting signal intensity of the released eTag was observed. The results illustrate the quantitative correlation between electrophoretic signal intensity and concentration of probe (Example 5; FIG. 3) and concentration of primary antibody (Example 6; FIGS. 4A-B).

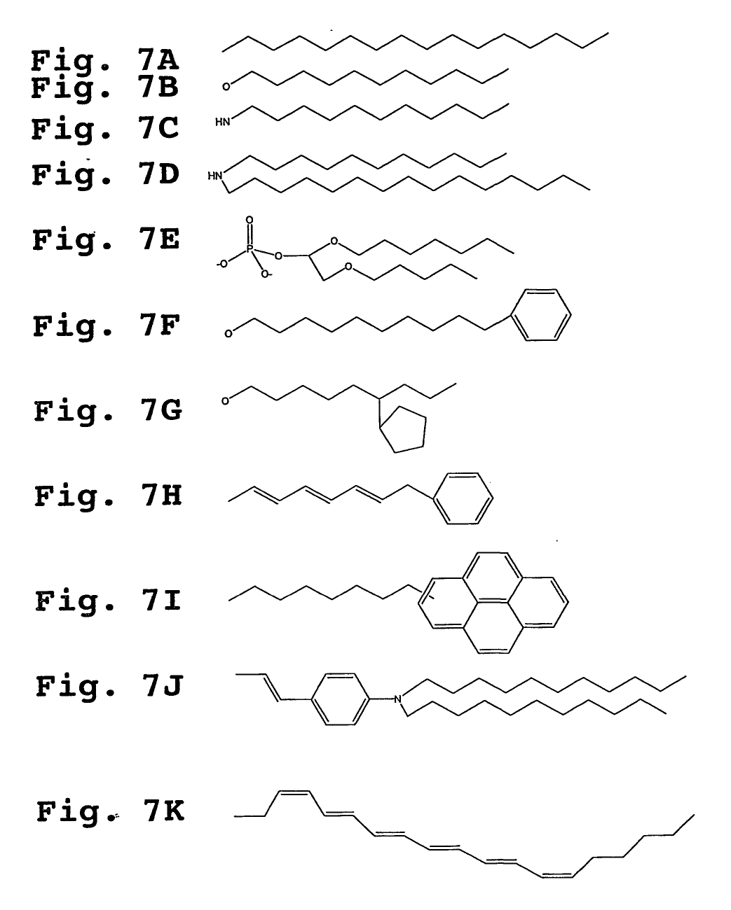

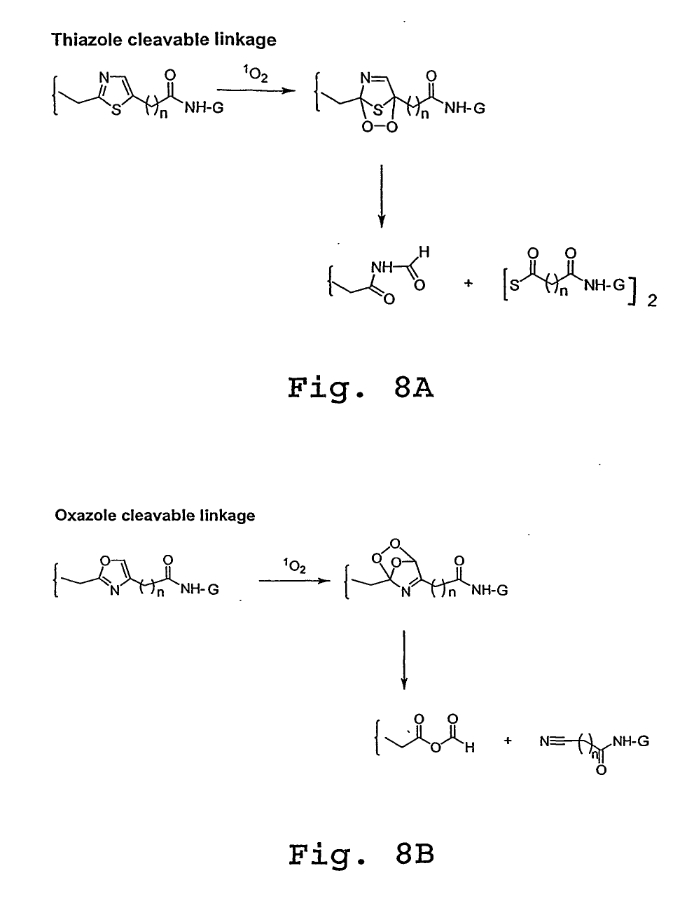

Example 7 describes a mixed cell assay, in which cells bearing a target receptor (e.g. HL-60 or Jurkat cells) were mixed with cells not bearing the receptor (mouse EL-4 cells). As shown in FIGS. 5-6, a direct correlation was observed between the electrophoretic signal and the number of cells in the mixture bearing the target receptor.

III. Electrophoretic Probes

The compounds of the invention, which may be referred to as “membrane anchored electrophoretic probes”, are of the general formula G-(L-E)ₘ, as noted above, where (G) is an anchoring group, preferably a lipophilic group, (L) is a cleavable linkage, and (E)ₘ represents one or more electrophoretic groups (E). By cleaving linkage L on probes bound to cells, electrophoretic tags E are released and can be detected. Each of these components is described in detail below.

A. The Lipophilic Anchoring Group (G)

The lipophilic group (G) is capable of binding to membranes such that all or substantially all probes of the invention are bound to the membranes in an assay solution, rather than being free in solution. The lipophilic moiety or functionality thus imparts lipophilicity or lipid solubility which decreases the wettability of surfaces by water and the solubility in water of compounds to which it is bound. The lipophilic moiety (G) can contain 1 to 50 or more atoms, usually carbon atoms substituted with hydrogen or halogen, and can include alkyl, alkene, alkyne, alkylidene, aryl and/or aralkyl. The lipophilic group or functionality will normally have one to six straight or branched chain alkyl groups of at least 5 carbon atoms, more usually at least 10 carbon atoms, and preferably at least 15 carbon atoms, not more than 50 carbon atoms, more usually not more than 30 carbon atoms. The alkyl group will normally be terminal and may be bonded to rings of 5 or 6 members, which may be alicyclic, heterocyclic, or aromatic. The lipophilic moiety may additionally be bonded to photosensitizers or chemiluminescent compounds.

The precursors to the lipophilic moiety can be alkyl compounds having a polar group that may be a single functionality or a complex group of functionalities on a hydrocarbon chain of the alkyl compound. The polar group can serve to link G with the cleavable linkage moiety (L). The polar group can be an acyl group, particularly a carboxy or phosphoryl ester; a hydroxylic group, which may be employed for forming an ether or ester link; an amino group, which may serve to provide an alkylamino, an amide, amidine, or urea link, or a mercaptan, which may serve to form a thioether group with an activated olefin, and the like. The lipophilic moiety is preferably linked to the cleavable linkage moiety (L) via an alkyl (CH₂), amide, sulfonamide, carboxyamide, carboxylate ester, urethane, urea, or thiourea linkage. The lipophilic moiety can thus be chosen from the large variety of potential fatty acid components, such as, for example, myristic acid, palmitic acid, or stearic acid or a substituted or unsubstituted sphingosine.

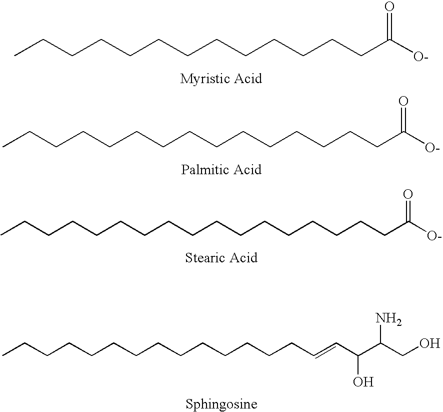

Lipophilic moieties can also include cholesterol; other steroids, including progestagens such as progesterone, glucocorticoids such as cortisol, mineralocorticoids such as aldosterone, androgens such as testosterone and androstenedione, and estrogens such as estrone and estradiol; glycolipids such as cerebroside or ganglioside; molecules having isoprenoid side chains such as vitamin K₂, coenzyme Q₁₀, chlorophyll, or carotenoids; low density lipoprotein (LDL), and the like. Generally, any sterol capable of attachment or which can be modified for attachment to the cleavable linkage (L) may be used in the practice of the present invention. For example, such sterols include but are not limited to cholesterol, vitamin D, phytosterols (including but not limited to sitosterol, campesterol, stigmasterol, and the like), steroid hormones, and the like.

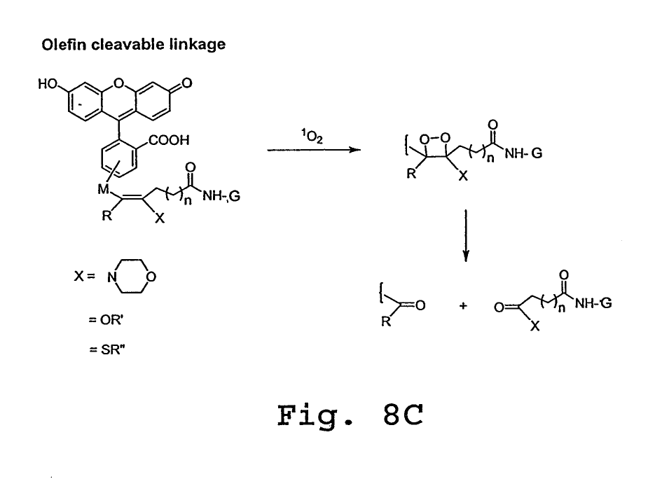

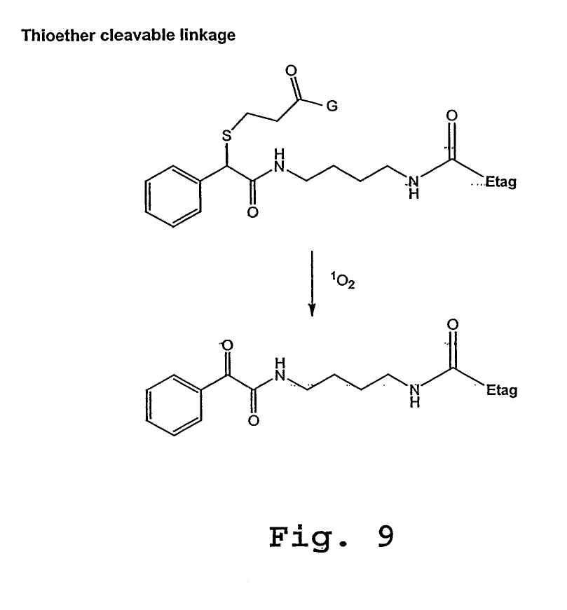

In one aspect of the invention, G represents an amphiphilic compound, particularly a phospholipid. The phospholipids are based upon alkyl carboxylic acid esters of alkyl polyols, where at least one hydroxylic group is substituted with a carboxylic acid ester, and where the alkyl group is as defined above. The alkyl group can thus have from about 1 to 50, more usually from about 10 to 20 carbon atoms, which may have from 0 to 5, more usually from 0 to 2, sites of ethylenic saturation and at least one hydroxyl group substituted with phosphate to form a phosphate ester. The phosphate group may be further substituted with small aliphatic compounds which are of di or higher functionality, and generally having hydroxyl or amino groups. Thus, the lipophilic moiety may include phospholipids such as phosphatidyl choline, phosphatidyl serine, phosphatidyl ethanolamine, phosphatidyl inositol, or cardiolipin, and sphingolipids such as sphingomyelin Representative lipophilic groups are illustrated in FIGS. 7A-K.

In another aspect, G can be a peptide, more particularly a lipophilic or a “greasy” peptide, that is capable of associating with a bilayer, such as membranes. A greasy peptide generally refers to any peptide whose affinity for lipid surfaces is measured by a dissociation constant of K_{d} about 10⁻⁶ or less. The ability of a greasy peptide to keep the probes anchored in the membrane can be verified empirically, or it can be predicted on the basis of a high proportion of lipophilic residues and relatively few charged residues using known algorithms. For example, use of a computer program that formulates a hydropathy scale from the amino acid sequence of a protein, utilizing the hydrophobic and hydrophilic properties of each of the 20 natural amino acids is described in Kyte et al., J. Mol. Biol. 157:105-132 (1982) and Hopp and Woods, Proc. Natl. Acad. Sci. 78:3824-3828 (1981). In one aspect, the greasy peptide can be a transmembrane domain that is capable of anchoring the compounds of the invention in the membranes. The transmembrane domain is thus sufficiently lipophilic to penetrate the lipid bilayer and keep the compounds inserted in the membrane in a stable fashion. Typically, the transmembrane domain will span the entire lipid bilayer one or more times. The transmembrane domain can be derived from the receptor under investigation, such as a G-protein coupled receptor, and can provide a second signal. Alternatively, artificially designed polypeptide sequences can be used for transmembrane domains in this invention.

B. The Cleavable Linkage (L)

Cleavable linkage, L, can be virtually any chemical linking group that may be cleaved under conditions that do not degrade the structure or affect detection characteristics of the released electrophoretic tag, E.

In a non-homogeneous format, labeled objects, such as biological cells, liposomes, or the like, with desired properties, are separated or isolated from objects not having such properties. Thus, a wide selection of cleavable linkages and cleavage agents are available for use with the invention. Cleavable linkages may include not only linkages that are labile to reaction with a locally acting reactive species, such as hydrogen peroxide, singlet oxygen, or the like, but also linkages that are labile to agents that operate throughout a reaction mixture, such as base-labile linkages, photocleavable linkages, linkages cleavable by reduction, linkages cleaved by oxidation, acid-labile linkages, peptide linkages cleavable by specific proteases, and the like. References describing many such linkages include Greene and Wuts, Protective Groups in Organic Synthesis, Second Edition (John Wiley & Sons, New York, 1991); Hermanson, Bioconjugate Techniques (Academic Press, New York, 1996); and Still et al., U.S. Pat. No. 5,565,324. Exemplary cleavable linkages are illustrated in Table 1.

TABLE 1

| Linking Group | Cleavage Reagent |
|---|---|
| silyl | fluoride or acid |
| A (see below) | hv |
| B | Ce(NH₄)₂(NO₃)₆ |
| —NCO₂— | HO⁻, H⁺, or LiAlH₄ |
| C | O₃, OsO₄/IO₄⁻, or KMnO₄ |
| D | 1) O₂ or Br₂, MeOH |
| | 2) H₃O⁺ |
| —Si— | oxidation, H⁺, Br₂, Cl₂, etc. |
| E | H₃O⁺ |
| F | H₃O⁺ |
| G | F⁻ or H⁺ |
| H, where x is a keto, ester, | base, HO⁻ |
| amide, NO₂, sulfide, sulfoxide, | |
| sulfone, and related electron | |
| withdrawing groups. | |
| I | H₃O⁺ or reduction (e.g. Li/NH₃) |
| J | (Ph₃P)₃RhCl(H) |
| K | Li, Mg, or BuLi |
| M | Hg⁺² |
| N, where x is halogen or | Zn or Mg |
| pseudohalogen | |
| O | oxidation (e.g. Pb(OAc)₄ or |
| | H₃IO₆) |
| P, where X is a electron | base |
| withdrawing group | |

Illustrative cleavable linking groups and cleavage reagents referred to in Table I are shown below, where (L) shows the point of attachment of the electrophoretic tag (E).

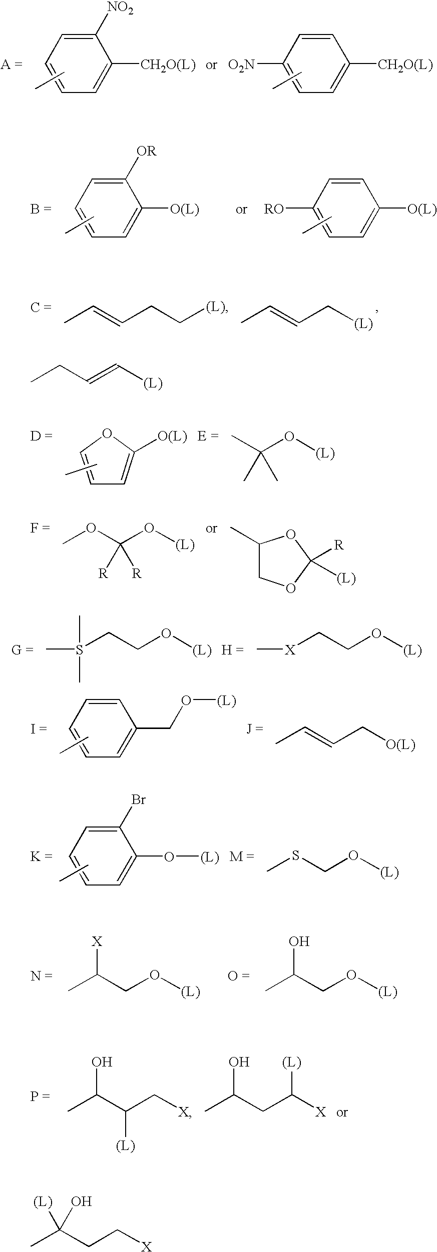

In one aspect, commercially available cleavable reagent systems may be employed with the invention. For example, a disulfide linkage may be introduced between a lipophilic anchor and an electrophoretic tag using a heterobifunctional agent such as N-succinimidyl 3-(2-pyridyldithio)propionate (SPDP), succinimidyloxycarbonyl-α-methyl-α-(2-pyridyldithio)toluene (SMPT), or the like, available from vendors such as Pierce Chemical Company (Rockford, Ill.). Disulfide bonds introduced by such linkages can be cleaved by treatment with a reducing agent, such as dithiothreitol (DTT), dithioerythritol (DTE), 2-mercaptoethanol, sodium borohydride, or the like. Typical concentrations of reducing agents to effect cleavage of disulfide bonds are in the range of from 10 to 100 mM. An oxidatively labile linkage may be introduced between a lipophilic anchor and an electrophoretic tag using the homobifunctional NHS ester cross-liking reagent, disuccinimidyl tartrate (DST) (also available from Pierce), which contains central cis-diols that are susceptible to cleavage with sodium periodate (e.g., 15 mM periodate at physiological pH for 4 hours). Linkages that contain esterified spacer components may be cleaved with strong nucleophilic agents, such as hydroxylamine, e.g. 0.1 N hydroxylamine, pH 8.5, for 3-6 hours at 37° C. Such spacers can be introduced by a homobifunctional cross-linking agent, such as ethylene glycol bis (succinimidylsuccinate) (EGS) available from Pierce (Rockford, Ill.). A base labile linkage can be introduced with a sulfone group. Homobifunctional cross-linking agents that can be used to introduce sulfone groups in a cleavable linkage include bis[2-(succinimidyloxycarbonyloxy)ethyl]sulfone (BSOCOES), and 4,4-difluoro-3,3′-dinitrophenylsulfone (DFDNPS). Exemplary basic conditions for cleavage include 0.1 M sodium phosphate, adjusted to pH 11.6 by addition of Tris base, containing 6 M urea, 0.1% SDS, and 2 mM DTT, with incubation at 37° C. for 2 hours. Photocleavable linkages include those disclosed in Rothschild et al., U.S. Pat. No. 5,986,076.

In another aspect of the invention, the cleavage moiety, L, is an “enzyme-cleavable peptide,” which is a peptide comprising an amino acid sequence that is recognized by a peptidase. Enzyme-cleavable peptides, typically from about 2 to 20 amino acids in length, are of sufficient length to project above the surfaces of the lipid bilayers. Such peptides are well known to ordinarily skilled artisans and include, for example and without limitation, the amino acid sequences: Ala-Ala-, Ala-Ala-Pro-Val (SEQ ID NO: 1), Ala-Ala-Met-, Ala-Ala-Pro-Phe- (SEQ ID NO:3), Ala-Ala-Pro-Met- (SEQ ID NO:4), Ala-Ala-Arg, Ser-Ala-Ala-Arg- (SEQ ID NO:5), Ser-Ser-Ala-Ala-Arg- (SEQ ID NO:6), Ser-S carboxyl sugar-Ala-Ala-Arg- (SEQ ID NO:7), Ala-Ala-Asp-, Ser-Ala-Ala-Asp- (SEQ ID NO:8), Ser-Ser-Ala-Ala-Asp- (SEQ ID NO:9), Arg-Pro-Lys-Pro-Leu-Ala-Nva- (SEQ ID NO:10), Ser-Arg-Pro-Lys-Pro-Leu-Ala-Nva- (SEQ ID NO:11), Ser-Ser-Arg-Pro-Lys-Pro-Leu-Ala-Nva (SEQ ID NO:12), Pro-Cha-Gly-Nva-His-Ala-Dpa-NH₂ (SEQ ID NO:13), Pro-Leu-Gly-Leu-Dpa-Ala-Arg-NH₂ (SEQ ID NO:14), Pro-Cha-Gly-Nva-, Pro-Leu-Gly-Leu (SEQ ID NO:15), Gly-Pro-Arg, Leu-Pro-Arg, Glu-Gly-Arg, and Gly-Pro-Gln-Gly-Ile- (SEQ ID NO: 16).

When the membrane anchored electrophoretic probes are used in a homogeneous assay format, cleavable linkage, L, is cleaved by a cleavage agent that acts over a short distance, so that only cleavable linkages in its immediate proximity are cleaved. Typically, such an agent is activated by making a physical or chemical change to the reaction mixture so that the agent produces a short lived active species that diffuses to a cleavable linkage to effect cleavage. The cleavage agent is preferably attached to a binding moiety which targets the cleavage agent, prior to activation, to a particular site in the proximity of the membrane anchored electrophoretic probe, such as a receptor in the same membrane. In such embodiments, a cleavage agent is referred to herein as a “cleavage-inducing moiety,” which is discussed more fully below.

The cleavage-inducing moiety may be an enzyme, sensitizer, or the like. Typically, the short-lived active species is an oxidizing agent, such as singlet oxygen, superoxide anion, hydrogen peroxide, or the like, and cleavable linkage, L, is cleaved whenever the local concentration of such active species is sufficiently high. Such oxidatively labile linkages include thioethers, selenoethers, olefins as described herein, and the like.

In one embodiment, cleavable olefin linkages of the invention include moieties of the following structure: 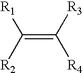
where each of R₁-R₄ is a carbon-containing group which may also contain a heteroatom such as oxygen, nitrogen, sulfur, or halogen, for example. The R₁-R₄ groups are independently selected from a wide variety of substituents. R₁-R₄ can be independently selected from the group consisting of hydrogen, alkyl, heteroalkyl, heteroaryl, heteroaralkyl, aryloxy, aryl, substituted aryl, hydroxyaryl or substituted hydroxyaryl, acyloxyaryl or substituted acyloxyaryl, silyloxyaryl or substituted siloxyaryl, aminoaryl or substituted aminoaryl, and sulfonamidoaryl or substituted sulfonamidoaryl. Any of the R₁-R₄ groups may be joined together to form one or more rings either on one side of the double bond, or across the double bond. The linking of the R groups may be through a bond, or through a bridge which may be a heteroatom or a carbon containing group optionally containing one or more heteroatoms.

Preferably, one of the R groups is an electron donating group, attached directly to the olefin or through conjugated double bonds. Enol ethers, enediol ethers, vinyl sulfides, enamines, and N-acylenamines are examples of olefins useful in the invention, where the heteroatom of the heteroalkyl substituent is attached directly to the double bond. Preferred embodiments of R₁-R₄ include aryl groups, such as phenyl and naphthyl groups, oxy groups of the formula YO—, wherein Y is aryl, alkyl, aralkyl, cycloalkyl, and the like, thio groups of the formula YS—, amino groups of the formula YHN—, and alkyl groups selected such that there is no proton in the allylic group position. Preferably, at least one R group is a YO— or YS— group, as the presence of the heteroatom promotes dioxetane formation. Each of the R groups may be substituted with optional substituents, including halogens, amines, alkoxy, heteroaryl, and the like. One or more of the R groups may comprise an auxiliary fluorophore, which may be selected from anthracenes, rhodamines, fluoescins, coumarins, erythrosins, acridines, pyrenes, stilbenes, nitrobenzoxadiazoles, quinolines, acidoacridines, carbazoles, fluorescent cyanines, carbocyanines, pyridinium salts, oxonols, resorting, and derivatives of these groups, as well as phenyl and naphthyl moieties.

Further, one of the R groups may bear a protective group which, if removed, induces decomposition of the dioxetane group formed from the precursor. Such groups include corresponding enzyme-labile substrates, such as phosphates, for example, and groups which may be removed by addition of non-enzymatic chemicals, such as base, electron donors, and the like, such as a silyl-protected OH group.

The lipophilic moiety (G) and the electrophoretic group (E) can be attached to the olefin directly, or via two of the R groups.

Thus, in one embodiment of the invention, L is a heteroatom-substituted olefin, wherein cleavage of a double bond to an oxo group releases the electrophoretic tag, E. Illustrative olefins include vinyl sulfides, vinyl ethers, enamines, imines substituted at the carbon atoms with an α-methine (CH, a carbon atom having at least one hydrogen atom), where the vinyl group may be in a ring, the heteroatom may be in a ring, or substituted on the cyclic olefinic carbon atom, and there will be at least one and up to four heteroatoms bonded to the olefinic carbon atoms. The resulting dioxetane may decompose spontaneously, by heating above ambient temperature, usually below about 75° C., by reaction with acid or base, or by photoactivation in the absence or presence of a photosensitizer. Such reactions are described in the following exemplary references: Adam and Liu, J. Amer. Chem. Soc. 94:1206-1209 (1972), Ando et al., J. C. S. Chem. Comm. 477-8 (1972), Ando et al., Tetrahedron 29:1507-13 (1973), Ando et al., J. Amer. Chem. Soc. 96:6766-8 (1974), Ando and Migita, ibid. 97: 5028-9 (1975), Wasserman and Terao, Tetrahedron Lett. 21:1735-38 (1975), Ando and Watanabe, ibid. 47:4127-30 (1975), Zaklika et al., Photochemistry and Photobiology 30:35-44 (1979), Adam et al., Tetrahedron Lett. 36:7853-4 (1995), and Hemmi et al., U.S. Pat. No. 5,756,726 (1998).

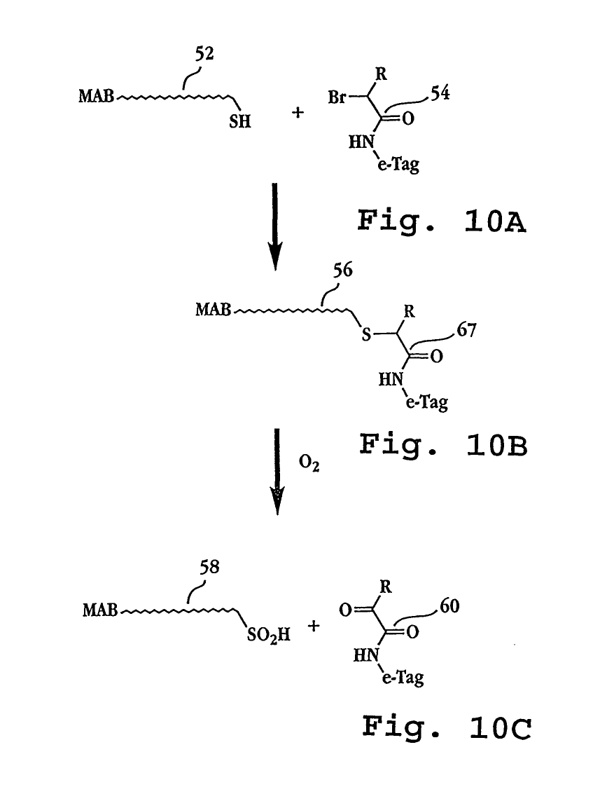

Reaction of singlet oxygen with an activated olefin as described herein, substituted with an electrophoretic moiety at one carbon atom and the lipophilic binding moiety at the other carbon atom of the olefin, results in formation of a dioxetane. See, for example, U.S. Pat. No. 5,807,675. These cleavable linkages may also be depicted by the following formula:
—W—(X)ₙC_{α}═C_{β}(Y)(Z)—
wherein W may be a bond, a heteroatom, e.g., O, S, N, P, or a metal that forms a stable covalent bond, or a functionality, such as carbonyl, imino, etc., and is bonded to X or C_{α}, and further (through the open valence shown) to an electrophoretic tag. See, for example, the structure of FIG. 8C, where a group M (mobility group) linked to a fluorescein derivative is one embodiment of W linked to an eTag.

At least one X is an aliphatic, aromatic, alicyclic or heterocyclic group bonded to C_{α} through a heteroatom, e.g., N, O, or S, and the other X may be the same or different and may in addition be hydrogen, aliphatic, aromatic, alicyclic or heterocyclic, usually being aromatic or aromatic heterocyclic; wherein one X may be taken together with Y to form a ring, usually a heterocyclic ring, with the carbon atoms to which they are attached. Generally, when other than hydrogen, X comprises from about 1 to 20, usually 1 to 12, more usually 1 to 8 carbon atoms; one X has 0 to 6, usually 0 to 4 heteroatoms, while the other X has at least one heteroatom and up to 6 heteroatoms, usually 1 to 4 heteroatoms;

Y comes within the definition of X, usually being bonded to C_{β} through a heteroatom, and, as indicated, may be taken together with X to form a heterocyclic ring;

Z is usually aromatic, including heterocyclic aromatic, comprising from about 4 to 12, usually 4 to 10 carbon atoms and 0 to 4 heteroatoms, as described above (i.e. O, S, N, P, or metal), and is bonded to C_{β} directly or through a heteroatom, as described above;

n is 1 or 2, depending upon whether the electrophoretic moiety is bonded to C_{α} or X;

and one of Y and Z has a functionality for binding to the lipophilic moiety, or is bound to the lipophilic moiety, e.g. by serving as, or including a linkage group, to a lipophilic moiety, G.

Preferably, W, X, Y, and Z are selected so that upon cleavage, the electrophoretic tag, E, is within the size limits described below.

While not depicted in the formula, there may be a plurality of electrophoretic moieties E in a single molecule, by having one or more electrophoretic moieties joined to one or both X's.

Illustrative cleavable linkages include S-(eTag)-3-thiolacrylic acid, N-(eTag)-N′-methyl-4-amino-4-butenoic acid, O-(eTag)-3-hydroxyacrolein, and N-(4-carboxyphenyl)-2-(eTag)-imidazole, oxazole, and thiazole.

Also of interest are N-alkyl acridinyl derivatives, substituted at the 9 position with a divalent group of the formula:
—(CO)X¹(A)-
wherein:

X¹ is a heteroatom selected from the group consisting of O, S, N, and Se, usually one of the first three; and

A is a chain of at least 2 carbon atoms and usually not more than 6 carbon atoms substituted with an electrophoretic tag reporter, where preferably the other valences of A are satisfied by hydrogen, although the chain may be substituted with other groups, such as alkyl, aryl, heterocyclic groups, etc., A generally being not more than 10 carbon atoms.

Also of interest are heterocyclic compounds, such as diheterocyclopentadienes, as exemplified by substituted imidazoles, thiazoles, oxazoles, etc., where the rings are generally substituted with at least one aromatic group.

Also of interest are tellurium (Te) derivatives, where the Te is bonded to an ethylene group having a hydrogen atom β to the Te atom, wherein the ethylene group is part of an alicyclic or heterocyclic ring, that may have an oxo group, preferably fused to an aromatic ring, and the other valence of the Te is bonded to the electrophoretic tag reporter. The rings may be coumarin, benzoxazine, tetralin, etc.

Several representative cleavable linkages and their cleavage products are illustrated in FIGS. 8A-C, where G represents a membrane anchoring group. Included are thiazole and oxazole cleavable linkages (FIGS. 8A-B), which may be represented as —CH₂-thiazole-(CH2)ₙ—C(═O)—NH— and —CH₂-oxazole-(CH2)ₙ—C(═O)—NH—, respectively, where n is preferably 1 to 12, and more preferably 1 to 6. In these figures, the open valence represents a linkage to an eTag.

Cleavage of the illustrated olefin linkage (FIG. 8C) results in an electrophoretic tag of the form R—(C═O)-M-D, where D is a detectable group (such as a fluorescein derivative, as shown in the Figure) and M is a mobility modifying group, as described above. R may be any substituent within the general description of the electrophoretic tags, E, provided below. Preferably, R is an electron-donating group, e.g. Ullman et al., U.S. Pat. No. 6,251,581; Smith and March, March's Advanced Organic Chemistry: Reactions, Mechanisms, and Structure, 5^{th} Edition (Wiley-Interscience, New York, 2001); and the like. More preferably, R is an electron-donating group having from 1-8 carbon atoms and from 0 to 4 heteroatoms selected from the group consisting of O, S, and N. In further preference, R is —N(Q)₂, —OQ, p-[C₆H₄N(Q)₂], furanyl, n-alkylpyrrolyl, 2-indolyl, or the like, where Q is alkyl or aryl. X is preferably morpholino, —OR′, or —SR″, where R′ and R″ are aliphatic, aromatic, alicyclic or heterocyclic groups having from 1 to 8 carbon atoms and 0 to 4 heteroatoms selected from the group consisting of O, S, and N. Note that substituents “X” and “R” are equivalent to substituents “X” and “Y” of the above formula describing cleavable linkage L. Cleavage by singlet oxygen proceeds via a dioxetane intermediate, as described above.

A preferred thioether cleavable linkage is illustrated in FIG. 9. Thioether cleavable linkages may be attached to various groups, such as membrane anchoring groups, G, or electrophoretic tags, E, by way of precursor compounds such as shown FIG. 10; see also FIGS. 11A-C.

C. The Electrophoretic Tag (E)

The electrophoretic tag, E, is a water soluble compound that is stable with respect to the conditions employed for cleavage of linkage L, especially singlet oxygen generation, and that includes a detection or reporter group. Otherwise, E may vary widely in size and structure. Preferably, E carries a charge at neutral pH and has a molecular weight in the range of from about 150 to about 10,000 daltons, more preferably, from about 150 to about 5000 daltons, and most preferably, from about 150 to 2500 daltons. Preferred structures of E are described more fully below. Preferably, the detection group generates an electrochemical, fluorescent, or chromogenic signal. Most preferably, the detection group generates a fluorescent signal.

Compositions of the invention include probes which comprise pluralities of electrophoretic tags that may be used together to carry out the multiplexed assays of the invention. Preferably, the plurality of electrophoretic tags in a composition is at least 5, and more preferably, at least 10. Still more preferably, the plurality of electrophoretic tags is in the range of from 5 to 200, and more preferably, from 5 to 100, or 5 to 75, or from 5 to 50, or from 10 to 30. Preferably, electrophoretic tags within a plurality of a composition each have either a unique charge-to-mass ratio and/or a unique optical property with respect to the other members of the same group of tags. Preferably, the optical property is a fluorescence property, such as emission spectrum, fluorescence lifetime, or the like. More preferably, the fluorescence property is an emission spectrum. For example, each electrophoretic tag of a plurality of tags may have the same fluorescent emission properties, but each will differ from the others by virtue of unique charge-to-mass ratios. On the other hand, two or more of the electrophoretic tags of a plurality of tags may have identical charge-to-mass ratios, but they will have unique fluorescent properties, e.g. spectrally resolvable emission spectra, so that all the members of the plurality are distinguishable by the combination of electrophoretic separation and fluorescence measurement.

Preferably, electrophoretic tags in a plurality of tags are detected by electrophoretic separation and fluorescence. Preferably, electrophoretic tags having substantially identical fluorescence properties have different electrophoretic mobilities so that distinct peaks in an electropherogram are formed under separation conditions. A measure of the distinctness, or lack of overlap, of adjacent peaks is electrophoretic resolution, which can be defined, in one of many ways, as the distance between adjacent peak maximums divided by four times the larger of the two standard deviations of the peaks. Preferably, adjacent peaks have a resolution, according to this definition, of at least 1.0, more preferably at least 1.5, and most preferably at least 2.0. In a given separation and detection system, the desired resolution may be obtained by selecting a plurality of electrophoretic tags whose members have electrophoretic mobilities that differ by at least a peak-resolving amount, such quantity depending on several factors well known to those of ordinary skill, including the signal detection system, the nature of the fluorescent moieties, the diffusion coefficients of the tags, the presence or absence of sieving matrices, nature of the electrophoretic apparatus, e.g. presence or absence of channels, length of separation channels, and the like.

Preferably, pluralities of electrophoretic tags of the invention are separated by a conventional capillary electrophoresis apparatus, either in the presence or absence of a conventional sieving matrix. Exemplary capillary electrophoresis apparatus include Applied Biosystems (Foster City, Calif.) models 310, 3100 and 3700; Beckman (Fullerton, Calif.) model P/ACE MDQ; Amersham Biosciences (Sunnyvale, Calif.) MegaBACE 1000 or 4000; SpectruMedix genetic analysis system; and the like. Preferably, in such a conventional apparatus, the electrophoretic mobilities of a plurality of electrophoretic tags differ by at least one percent, and more preferably, by at least a percentage in the range of from 1 to 10 percent. Electrophoretic mobility is proportional to q/M^{2/3}, where q is the charge on the molecule and M is the mass of the molecule. Desirably, the difference in mobility under the conditions of the determination between the closest electrophoretic labels will be at least about 0.001, usually 0.002, more usually at least about 0.01, and may be 0.02 or more.

A preferred structure of electrophoretic tag, E, is (M,D), where M is a mobility-modifying moiety and D is a detection moiety. The notation “(M,D)” is used to indicate that the ordering of the M and D moieties may be such that either moiety can be adjacent to the cleavable linkage, L. That is, “G-L-(M, D)” designates electrophoretic probe of either of two forms: “G-L-M-D” or “G-L-D-M.”

C1. Detection Moiety D

Detection moiety, D, may be a fluorescent label or dye, a chromogenic label or dye, an electrochemical label, or the like. Preferably, D is a fluorescent dye. Exemplary fluorescent dyes for use with the invention include water-soluble rhodamine dyes, fluoresceins, 4,7-dichlorofluoresceins, benzoxanthene dyes, and energy transfer dyes, disclosed in the following references: Handbook of Molecular Probes and Research Reagents, 8th ed., (Molecular Probes, Eugene, 2002); Lee et al., U.S. Pat. No. 6,191,278; Lee et al., U.S. Pat. No. 6,372,907; Menchen et al., U.S. Pat. No. 6,096,723; Lee et al., U.S. Pat. No. 5,945,526; Lee et al., Nucleic Acids Research, 25: 2816-2822 (1997); Hobb, Jr., U.S. Pat. No. 4,997,928; Khanna et al., U.S. Pat. No. 4,318,846; Reynolds, U.S. Pat. No. 3,932,415; Eckert et al., U.S. Pat. No. 2,153,059; Eckert et al., U.S. Pat. No. 2,242,572; Taing et al., International patent publication WO 02/30944; and the like. Further specific exemplary fluorescent dyes include 5- and 6-carboxyrhodamine 6G; 5- and 6-carboxy-X-rhodamine, 5- and 6-carboxytetramethylrhodamine, 5- and 6-carboxyfluorescein, 5- and 6-carboxy-4,7-dichlorofluorescein, 2′,7′-dimethoxy-5- and 6-carboxy-4,7-dichlorofluorescein, 2′,7′-dimethoxy-4′,5′-dichloro-5- and 6-carboxyfluorescein, 2′,7′-dimethoxy-4′,5′-dichloro-5- and 6-carboxy-4,7-dichlorofluorescein, 1′,2′,7′,8′-dibenzo-5- and 6-carboxy-4,7-dichlorofluorescein, 1′,2′,7′,8′-dibenzo-4′,5′-dichloro-5- and 6-carboxy-4,7-dichlorofluorescein, 2′,7′-dichloro-5- and 6-carboxy-4,7-dichlorofluorescein, and 2′,4′,5′,7′-tetrachloro-5- and 6-carboxy-4,7-dichlorofluorescein. Most preferably, D is a fluorescein or a fluorescein derivative.

In another aspect, the detection moiety of (MD) generates a fluorescent signal by an energy transfer mechanism. Preferably, in this aspect, D has the form “D₁-g-D₂” where D₁ and D₂ are acceptor-donor pairs of molecules, e.g. Wu et al., Anal. Biochem. 218:1-13 (1994), and g is a rigid linker that maintains D₁ and D₂ at a substantially constant distance. Guidance in selecting rigid linker, g, may be found in Wu et al. (cited above) and in U.S. Pat. Nos. 5,863,727; 5,800,996; 5,945,526; and 6,008,379. Either D₁ or D₂ may be the acceptor and the other the donor molecule in the pair. Exemplary energy transfer detection moieties for use with the invention are disclosed in Lee et al., U.S. Pat. No. 5,945,526; Lee et al., Nucleic Acids Research 25:2816-2822 (1997); Taing et al., PCT Publication WO 02/30944; and like references. Preferably, rigid linker, g, is selected so that the distance between D₁ and D₂ is maintained at a substantially constant distance within the range of from 10-100 Angstroms. A wide variety of linking groups may be employed with the proviso that the linkage is stable to the presence of singlet oxygen. Preferably, D₁ and D₂ are selected from the set of fluorescein, rhodamine, rhodamine 6G, rhodamine 110, rhodamine X, tetramethylrhodamine, and halogenated derivatives thereof. More preferably, D₁ and D₂ are both fluorescein dyes.

In one aspect, g may be selected from any of R₁-R₂-R₁ and R₁-R₂—C(═O)—X₁—R₃, the latter being present in either orientation with respect to D₁ and D₂; where X₁ is O, S, or NH; R₁ is (C₁-C₅ alkyldiyl, X₁, C(═O)) such that the moieties in parentheses are arranged in any linear order; R₂ is a 5 to 6 membered ring selected from the group consisting of cyclopentene, cyclohexene, cyclopentadiene, cyclohexadiene, furan, pyrrole, isopyrole, isoazole, pyrazole, isoimidazole, pyran, pyrone, benzene, pyridine, pyridazine, pyrimidine, pyrazine oxazine, indene, benzofuran, thionaphthene, indole and naphthalene; and R₃ is C₁-C₅ alkyldiyl.

As described above, each e-tag moiety E typically contains a detectable label D. Alternatively, an e-tag moiety may contain a functionality allowing it to bind to a label D after reaction with a sample is complete. In some instances, the detectable label may be part of the reagent cleaving the cleavable bond L. In one embodiment, a plurality of different functionalities are used for different binding members, for reaction with a label, and the different labels have corresponding functionalities that react with one of the first functionalities. For example, where the first functionalities include thiols, carboxyl groups, aldehydes and olefins, the labels could include activated olefins, alcohols, amines and thiol groups, respectively. By employing removable protective groups for one or more of the functionalities, the protective groups may be removed stepwise and the labels added stepwise, to avoid cross-reactivity.

C2. Mobility Modifier M

M is generally a chemical group or moiety having a particular charge-to-mass ratio and thus a particular electrophoretic mobility in a defined electrophoretic system. In a set of n electrophoretic probes, each unique mobility modifier may be designated Mⱼ, where j=1 to n, and n has a value as described above. That is, n is generally from 5 to 200, and more preferably, from 5 to 100, or 5 to 75, or from 5 to 50, or from 10 to 30.

The mobility-modifying moiety may be considered to include a mass-modifying region and/or a charge-modifying region or a single region that acts as both a mass- and charge-modifying region. In the probe sets utilized in the invention, the mobility-modifying moiety may have one or more of the following characteristics: (i) a unique charge-to-mass ratio due to variations in mass, but not charge; (ii) a unique charge-to-mass ratio due to changes in both mass and charge; and (iii) a unique charge-to-mass ratio of between about −0.0001 and about 0.5, usually, about −0.001 and about 0.1. As noted above, D is typically the same among a set or plurality of different electrophoretic probes, but may also differ among probe sets, contributing to the unique electrophoretic mobilities of the released electrophoretic tag.

The size and composition of mobility-modifying moiety, M, can vary from a bond to about 100 atoms in a chain, usually not more than about 60 atoms, and more usually not more than about 30 atoms, where the atoms are preferably selected from carbon, oxygen, nitrogen, phosphorous, boron and sulfur. Generally, when other than a bond, the mobility-modifying moiety has from about 0 to about 40, more usually from about 0 to about 30, heteroatoms, which in addition to the heteroatoms indicated above may include halogen or another heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (including ammonium), phosphonium, sulfonium, oxonium, etc., where substituents are generally aliphatic of from about 1-6 carbon atoms, the total number of carbon atoms per heteroatom usually being less than about 12, preferably less than about 9. Other substituents may include hydroxyl groups, including phenolic groups, carboxyl groups, esters, amides, phosphates, and heterocycles. The charged mobility-modifying moieties generally have only negative or only positive charges, although one may have a combination of charges, particularly where a region to which the mobility-modifying moiety is attached is charged and the mobility-modifying moiety has the opposite charge.

In various embodiments, M may an oligomer, having monomers of the same or different chemical characteristics, e.g., nucleotides and amino acids. The mobility-modifying moieties may comprise a single type of monomer that provides the different functionalities for oligomerization and that carries a charge. Alternatively, two or more different monomers may be employed. Substituted diols may be used, where the substituents are charged and dibasic acids. Illustrative of such oligomers is the combination of diols or diamino, such as 2,3-dihydroxypropionic acid, 2,3-dihydroxysuccinic acid, 2,3-diaminosuccinic acid, 2,4-dihydroxyglutaric acid, etc. The diols or diamino compounds can be linked by dibasic acids, which dibasic acids include the inorganic dibasic acids indicated above, as well as organic dibasic acids, such as oxalic acid, malonic acid, succinic acid, maleic acid, fumaric acid, carbonic acid, etc. Instead of using esters, amides may be used, and amino acids or diamines and diacids may be employed. Alternatively, the hydroxyls or amines may be linked with alkylene or arylene groups.

Pluralities of electrophoretic tags may include oligopeptides for providing the charge, particularly oligopeptides of from 2-6, usually 2-4 monomers, either positive charges, resulting from lysine, arginine and histidine, or negative charges, resulting from aspartic and glutamic acid. Unnatural or synthetic amino acids, such as such as taurine, phosphate substituted serine or threonine, S-α-succinylcysteine, can also be used, as well as co-oligomers of diamines and amino acids, etc.

In one aspect of the present invention, the charge-imparting moiety is conveniently composed primarily of amino acids but also may include thioacids and other carboxylic acids having from one to five carbon atoms. The charge imparting moiety may have from 1 to about 30, preferably 1 to about 20, more preferably, 1 to about 10 amino acids per moiety and may also comprise 1 to about 3 thioacids or other carboxylic acids. However, when used with an uncharged sub-region, the charged sub-region will generally have from 1 to about 4, frequently 1 to about 3 amino acids. Any amino acid, either naturally occurring and/or synthetic, may be employed.

The desired charge-to-mass ratio can be achieved by employing monomers that have substituents that provide charges, or which may be modified to provide charges. For example, the hydroxyl groups of serine or threonine may be modified with phosphate to provide negatively charged mobility-modifying moieties. With arginine, lysine and histidine, positively charged mobility-modifying moieties are provided. Oligomerization may be performed in conventional ways to provide the appropriately sized mobility-modifying moiety. The different mobility-modifying moieties may have different orders of oligomers, generally from 1 to 20 repeating units, more usually about 1 to 12, where a repeating unit may have from 1 to 2 different monomers. For the most part, oligomers are used with other than nucleic acid target-binding regions. The polyfunctionality of the monomeric units provides for functionalities at the termini that may be used for conjugation to other moieties, so that the available functionality for reaction may be converted to a different functionality. For example, a carboxyl group may be reacted with an aminoethylthiol, to provide an amide with a terminal thiol functionality for reaction with an activated olefin.

By using monomers that have about 1 to about 3 charges, a low number of monomers can be employed to provide for mobility variation with changes in molecular weight. Of particular interest are polyolpolycarboxylic acids having from about two to four of each functionality, such as tartaric acid, 2,3-dihydroxyterephthalic acid, 3,4-dihydroxyphthalic acid, D⁵-tetrahydro-3,4-dihydroxyphthalic acid, etc. To provide for an additional negative charge, these monomers may be oligomerized with a dibasic acid, such as a phosphoric acid derivative to form the phosphate diester. Alternatively, the carboxylic acids can be used with a diamine to form a polyamide, while the hydroxyl groups can be used to form esters, such as phosphate esters, or ethers such as the ether of glycolic acid, etc.

To vary mobility, various aliphatic groups of differing molecular weight may be employed, such as polymethylenes, polyoxyalkylenes, polyhaloaliphatic or aromatic groups, polyols, e.g., sugars, where the mobility will differ by at least about 0.01, more usually at least about 0.02 and more usually at least about 0.5. Methods of forming selected-length polyethylene oxide-containing chains are well known, see, e.g. Grossman et al., U.S. Pat. No. 5,777,096. It can be appreciated that these methods, which involve coupling of defined-size, multi-subunit polymer units to one another, directly or via linking groups, are applicable to a wide variety of polymers, such as polyethers (e.g., polyethylene oxide and polypropylene oxide), polyesters (e.g., polyglycolic acid, polylactic acid), polypeptides, oligosaccharides, polyurethanes, polyamides, polysulfonamides, polysulfoxides, polyphosphonates, and block copolymers thereof, including polymers composed of units of multiple subunits linked by charged or uncharged linking groups. In addition to homopolymers, the polymer chains used in accordance with the invention include selected-length copolymers, e.g., copolymers of polyethylene oxide units alternating with polypropylene units. Additionally, polypeptides of selected lengths and amino acid composition (i.e., containing naturally occurring or man-made amino acid residues), as homopolymers or mixed polymers may be used.

Various oligomers may be synthesized on a support or produced by cloning or expression in an appropriate host. Conveniently, polypeptides can be produced having only one cysteine or serine/threonine/tyrosine, aspartic/glutamic acid, or lysine/arginine/histidine, other than an end group, providing a unique functionality which may be differentially functionalized. By using protective groups, a side-chain functionality can be distinguished from a terminal amino acid functionality. Also, by appropriate design, one may provide for preferential reaction between the same functionalities present at different sites on the mobility-modifying moiety. Whether one uses synthesis or cloning for preparation of oligopeptides, is to a substantial degree dependent on the length of the mobility-modifying moiety.

(M, D) moieties can be conveniently constructed from one or more of the same or different common or commercially available linking, cross-linking, and labeling reagents that permit facile assembly, especially using a commercial DNA or peptide synthesizer for all or part of the synthesis. In this aspect, (M, D) moieties are made up of subunits usually connected by phosphodiester and amide bonds. Exemplary precursors that form amide bonds include Fmoc- or Boc-protected amino acid precursors, and derivatives thereof, e.g. as commercially available from AnaSpec, Inc. (San Jose, Calif.). Exemplary precursors that form phosphodiester bonds include, but are not limited to, dimethoxytrityl (DMT)-protected hexaethylene glycol phosphoramidite, 6-(4-monomethoxytritylamino)hexyl-(2-cyanoethyl)-(N,N-diisopropyl)-phosphoramidite, 12-(4-monomethoxytritylamino)dodecyl-(2-cyanoethyl)-(N,N-diisopropyl)-phosphoramidite, 2-[2-(4-monomethoxytrityl)aminoethoxy]ethyl-(2-cyanoethyl), N,N-diisopropyl)-phosphoramidite, (S-trityl-6-mercaptohexyl)-(2cyanoethyl)-(N,N-diisopropyl)-phosphoramidite, 5′-fluorescein phosphoramidite, 5′-hexachloro fluorescein phosphoramidite, 5′-tetrachloro fluorescein phosphoramidite, 9-O-dimethoxytrityl-triethylene glycol, 1-[(2-cyanoethyl)-(N,N-diisopropyl)]-phosphoramidite, 3-(4,4′-dimethoxytrityloxy)propyl-1-[(2-cyanoethyl)-(N,N-diisopropyl)]-phosphoramidite, 5′-O-dimethoxytrityl-1′,2′-dideoxyribose-3′-[(2-cyanoethyl)-(N,N-diisopropyl)]-phosphoramidite, 18-O-dimethoxytrityl hexaethyleneglycol, 1-[(2-cyanoethyl)-(N,N-diisopropyl)]-phosphoramidite, 12-(4,4′-dimethoxytrityloxy)dodecyl-1-[(2-cyanoethyl)-(N,N-diisopropyl)]-phosphoramidite, 1,3-bis-[5-(4,4′-dimethoxytrityloxy) pentylamido]propyl-2-[(2-cyanoethyl)-(N,N-diisopropyl)]-phosphoramidite, 1-[5-(4,4′-dimethoxytrityloxy)pentylamido]-3-[5-fluorenomethoxycarbonyloxypentylamido]-propyl-2-[(2-cyanoethyl)-(N,N-diisopropyl)]-phosphoramidite, Tris-2,2,2-[3-(4,4′-dimethoxytrityloxy)propyloxymethyl]ethyl-[(2-cyanoethyl)-(N,N-diisopropyl)]-phosphoramidite, succinimidyl trans-4-(maleimidylmethyl)cyclohexane-1-carboxylate (SMCC), succinimidyl 3-(2-pyridyldithio)propionate (SPDP), succinimidyl acetylthioacetate, Texas Red-X-succinimidyl ester, 5- and 6-carboxy tetramethylrhodamine succinimidyl ester, bis-(4-carboxypiperidinyl)sulfonerhodamine di(succinimidyl ester), 5- and 6-((N-(5-aminopentyl)aminocarbonyl)tetramethylrhodamine, succinimidyl 4-(p-maleimidophenyl)butyrate (SMPB); N-γ-maleimidobutyryl-oxysuccinimide ester (GMBS); p-nitrophenyl iodoacetate (NPIA); 4-(4-N-maleimidophenyl)butyric acid hydrazide (MPBH); and like reagents. Accordingly, mobility modifiers M may be constructed from such reagents. The above reagents are commercially available, e.g. from Glen Research (Sterling, Va.), Molecular Probes (Eugene, Oreg.), Pierce Chemical, and like reagent providers. Use of the above reagents in conventional synthetic schemes is well known in the art, e.g. Hermanson, Bioconjugate Techniques (Academic Press, New York, 1996).

In another aspect, (MD) moieties are constructed from chemical scaffolds used in the generation of combinatorial libraries. For example, the following references describe scaffold compounds useful in generating diverse mobility modifying moieties: peptoids (PCT Publication WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomeres such as hydantoins, benzodiazepines and dipeptides (Hobbs DeWitt, S. et al., Proc. Nat. Acad. Sci. U.S.A. 90: 6909-6913 (1993)), vinylogous polypeptides (Hagihara et al., J. Amer. Chem. Soc. 114: 6568 (1992)), nonpeptidal peptidomimetics with a β-D-glucose scaffolding (Hirschmann, R. et al., J. Amer. Chem. Soc. 114: 9217-9218 (1992)), analogous organic syntheses of small compound libraries (Chen, C. et al., J. Amer. Chem. Soc. 116:2661(1994)), oligocarbamates (Cho, C. Y. et al., Science 261: 1303(1993)), peptidyl phosphonates (Campbell, D. A. et al., J. Org. Chem. 59:658(1994)); Cheng et al., U.S. Pat. No. 6,245,937; Heizmann et al., “Xanthines as a scaffold for molecular diversity,” Mol. Divers. 2:171-174 (1997); Pavia et al., Bioorg. Med. Chem. 4:659-666 (1996); Ostresh et al., U.S. Pat. No. 5,856,107; Gordon, E. M. et al., J. Med. Chem. 37:1385 (1994); and the like. Preferably, in this aspect, D is a substituent on a scaffold and M is the rest of the scaffold.

In another aspect, mobility-modifying moiety, M, comprises an alkylene or aralkylene group, the latter comprising a divalent aliphatic group having about 1 to about 2 aliphatic regions and about 1 to about 2 aromatic regions, generally benzene, where the groups may be substituted or unsubstituted, usually unsubstituted, comprising from 2 to about 16, more usually 2 to about 12, carbon atoms. The mobility-modifying moiety may be used to link one or more fluorescers to a monomeric unit, e.g., a nucleotide. The mobility-modifying moiety may terminate in a carboxy, hydroxy or amino group, forming an ester or amide upon conjugation. By varying the substituents on the fluorescer(s), one can vary the mass in units of at least about 5 or more, usually at least about 9. To provide further variation, a thiosuccinimide group may be employed to join alkylene or aralkylene groups, such that the total number of carbon atoms may be in the range of 2 to about 30, more usually 2 to about 20. Instead of or in combination with the above groups, and to add hydrophilicity, alkyleneoxy groups may be used.

In some embodiments, the electrophoretic moieties need not be charged, but merely differ in mass. Thus, the same or similar monomers can be used, where the functionalities are neutral or converted to neutral moieties, such as esters and amides of carboxylic acids. Also, the electrophoretic moieties may be varied by isotopic substitution, such as ²H, ¹⁸O, ¹⁴C, etc.

Diversity in sets of probes or e-tags can also be achieved via the chemical and optical characteristics of the label, the use of energy transfer complexes, and variations in the chemical nature of the mobility-modifying moiety which affect mobility, e.g. via folding, interaction with the solvent and ions in the solvent, and the like.

In one embodiment, a probe G-L-M-D may be represented by the formula:
G-L-(amino acid)ₙ-L=-Fluorescer
wherein L=is a bond or a linking group of from 1 to 20 atoms other than hydrogen, n is 1 to 20, and L is a cleavable linkage to the polypeptide-binding moiety. In this embodiment, G is linked to the terminal amino acid by a cleavable linkage. An example of this embodiment, by way of illustration and not limitation, is one in which the fluorescer is fluorescein, L=is a bond in the form of an amide linkage involving the meta-carboxyl of the fluorescein and the terminal amine group of lysine, and G is a polypeptide-binding moiety.

In another embodiment, after release, electrophoretic tag, E, is defined by the formula:
(A-M-D)
wherein:

A is —C(═O)R, where R is an aliphatic, aromatic, alicyclic or heterocyclic group having from 1 to 8 carbon atoms and 0 to 4 heteroatoms selected from the group consisting of O, S, and N; —CH₂—C(═O)—NH—CHO; —SO₂H; —CH₂—C(═O)O—CHO; —C(═O)NH—(CH₂)ₙ—NH—C(═O)C(═O)—(C₆H₅), where n is in the range of from 2 to 12;

D is a fluorescent dye; and

M is a mobility modifier as described above, with the proviso that the total molecular weight of A-M-D be within the range of from about 150 to about 5000 daltons.

In a preferred aspect, D is a fluorescein and the total molecular weight of A-M-D is in the range of from about 150 to about 2500 daltons.

In another preferred aspect, D is of the form “D₁-g-D₂” as described above.

D. Multiple Electrophoretic Tags

It may be advantageous to effect the release of multiple electrophoretic tag reporters for a binding event involving an individual target molecule. In a sense, this results in an amplification of signal. Where the lipophilic moiety has a plurality of sites for attachment such as, for example, multiple functional groups to which L-E can be attached, a plurality of electrophoretic tags can be attached to provide compounds of the structure G-(L-E)ₘ. For example, each lipophilic moiety (G) can have attached L-E moieties that result in the release of from 2-1000, preferably from 2-300, and more preferably from 2-100, and still more preferably from about 2 to about 10 molecules of detectable moieties per molecule. Each electrophoretic tag (E) released can be the same or can be different such that each different E is detectable.

In another aspect of the invention, the electrophoretic tag moieties are cleavably attached to a hub, to which the lipophilic moiety (G) is also attached. The hub nucleus can be a polyfunctional material, normally polymeric, having a plurality of functional groups, e.g., hydroxy, amino, mercapto, carboxy, ethylenic, aldehyde, etc., as sites for linking. The functionalities on the hub should be those that are reactive with a functionality on the L-E moiety or the G moiety to be attached. Some functionalities are preferred over others because of their ability to resist participation in unwanted side reactions. The hub nucleus is usually at least about 35,000 molecular weight and may be about 10 million or more molecular weight, but usually under about 600,000, more usually under about 300,000. Illustrative hub nuclei include polysaccharides, polypeptides, such as polylysine, polynucleotides, ion exchange resins, and the like. The hub is in one aspect a branched linker, which has multiple sites for attachment of the L-E moieties. Thus, the hub has an attachment site for attaching the lipophilic moiety and a plurality of sites for attachment of a plurality of L-E moieties. In another aspect, such a branched linker may comprise a streptavidin, or like polyvalent molecule, that is bound to a biotin which is covalently linked to a lipophilic moiety, G. Multiple electrophoretic tags can be attached by cleavable linkages to a biotinylated polymeric backbone, such as amino-dextran, which is then attached to the membrane-bound streptavidin through an available biotin binding site.

In one embodiment, the hub nucleus is a hydrophilic polymer, generally, an addition or condensation polymer with multiple functionality to permit the attachment of multiple moieties. One class of polymers that is useful for the reagents of the present invention comprises the polysaccharide polymers. Polysaccharides such as dextrans, sepharose, polyribose, polyxylose, and the like may be used. Another class of polymers are those that result from the addition polymerization of substituted ethylene or butadiene type monomers, including short chain unsaturated monomers such as propylene, wherein these monomers have substituents that are hydrophilic groups or can be derivatized to hydrophilic groups. Suitable hydrophilic groups that may be attached to the ethylene include hydroxy, carboxy and the ester and amides thereof, amines, and the like. If acrylic acid monomers are used, the acid can be derivatized to suitable reactive groups prior to or subsequent to polymerization. Thus, for example, the ester formed from ethylene glycol and acrylic acid provides a hydroxyl group for derivatization to the components of the e-tag probe. Other suitable polymers include polyallyl amines and alcohols such as, for example, polyvinyl alcohol. In addition to utilizing polymers derived from a single monomer, mixed polymers may also be employed. In this case, the hydrophilicity may be provided by a non-reactive component such as polyethylene glycol, which is then further polymerized to monomers that bear the appropriate functional groups for reaction with the components of the e-tag probe. One such polymer is a copolymer of polyethylene glycol with polyvinyl alcohol. One specific example of a hub is dextran, to which about 10 to about 300 molecules of e-tag moieties may be attached per one molecule of dextran.

Accordingly, in the present invention one or more hub molecules can be attached to a lipid bilayer by means of the lipophilic moiety (G). The electrophoretic tag moieties can be attached to the hub by means of a cleavable linkage. Upon exposure to a cleavage-inducing reagent, multiple electrophoretic tag reporters are released for subsequent detection. Depending upon the reagent to which the electrophoretic tag moiety is attached, as discussed above, there may be a single electrophoretic tag moiety or a plurality of electrophoretic tag moieties, generally ranging from about 1 to about 10⁵, more usually ranging from about 1 to about 300, more particularly ranging from about 1 to about 20. The number of electrophoretic tag moieties attached to a lipid bilayer depends upon the sensitivity required, the solubility of the electrophoretic tag moiety, the effect on the assay of a plurality of electrophoretic tag moieties, and the like.

IV. Cleavage Agents

As mentioned above, cleavable linkage, L, is cleaved by a cleavage agent that may vary widely depending on several factors, including the chemical nature of the cleavable linkage, whether other assay components are stable in the presence of the cleavage agent, whether the membrane anchored electrophoretic probes are used in a homogeneous or non-homogeneous assay format, and the like. In a non-homogeneous assay format, where the linkages sought to be cleaved are separated from those that are intended to remain intact, cleavage agents may include acids, bases, oxidants, including singlet oxygen, hydrogen peroxide, and the like, reductants, light, enzymes, including proteases and nucleases, nucleophilic reagents, and the like. In such formats, after separation, selection of cleavage reagent depends on the chemical nature of the cleavable linkage, the stability of the released electrophoretic tag in the presence of the cleavage agent, the affect of side products created in the cleavage step on the electrophoretic separation and detection of the electrophoretic tags, and the like.

Generally, in a non-homogeneous format, the cleavage agent, such as a photosensitizer, chemical reagent or enzyme, need not be attached to a binding agent to localize its action. In a homogeneous format, on the other hand, a cleavage agent must act only in the proximity of the cleavable linkages intended to be cleaved. Preferably, this is accomplished by attaching a cleavage agent to a binding agent, such as an antibody, antibody binding composition, or the like. As used herein, the term “cleavage-inducing moiety” refers to a cleavage agent that is attached to a binding agent for the purpose of localizing the cleavage effects of the cleavage agent.

In one aspect, a cleavage-inducing moiety is a group that produces an active species that is capable of cleaving a cleavable linkage, preferably by oxidation. Preferably, the active species is a chemical species that exhibits short-lived activity so that its cleavage-inducing effects occur only in the proximity of the site of its generation. Either the active species is inherently short lived, so that it will not create significant background beyond the proximity of its creation, or a quencher compound is employed that efficiently reacts with the active species, so that it is not available to react with cleavable linkages beyond a short distance from the site of its generation. Illustrative active species include singlet oxygen, hydrogen peroxide, NADH, hydroxyl radicals, phenoxy radical, superoxide, and the like. Illustrative quenchers for active species that cause oxidation include polyenes, carotenoids, vitamin E, vitamin C, amino acid-pyrrole N-conjugates of tyrosine, histidine, and glutathione, and the like, e.g. Beutner et al., Meth. Enzymol., 319: 226-241 (2000).

An important consideration for the cleavage-inducing moiety and the cleavable linkage is that they not be so far removed from one another when bound to a target that the active species generated by the sensitizer diffuses and loses its activity before it can interact with the cleavable linkage. Accordingly, a cleavable linkage is preferably within 1000 nm, preferably 20-100 nm, of a bound cleavage-inducing moiety. This effective range of a cleavage-inducing moiety is referred to herein as its “effective proximity.”

Generators of active species include enzymes, such as oxidases, e.g. glucose oxidase, xanthene oxidase, D-amino acid oxidase, NADH-FMN oxidoreductase, galactose oxidase, glyceryl phosphate oxidase, sarcosine oxidase, choline oxidase and alcohol oxidase, which produce hydrogen peroxide, horse radish peroxidase, which produces hydroxyl radical, various dehydrogenases that produce NADH or NADPH, and urease, which produces ammonia to create a high local pH.

One type of cleavable linkage is based on the oxidation of sulfur or selenium, where a thioether, sulfoxide, or selenium analog thereof, as described above, is present at the α- or β-position in relation to an activating group, which renders the hydrogen α to the activating group acidic and capable of being removed by base, so as to release the oxidized functionality to which is attached the releasable portion of the e-tag, or to be subject to oxidation with release of the e-tag.

Alternatively, one may use metal chelates that are stable at one oxidation state and unstable at another oxidation state. Other compounds include α-substituted methylquinones which have the releasable portion of a reagent bonded through a leaving group, such as sulfonyl, oxy, amino, etc.

A sensitizer is a molecule, usually a compound, that can be induced to generate a reactive intermediate, or species, usually singlet oxygen. Preferably, a sensitizer used in accordance with the invention is a photosensitizer. However, other sensitizers can be employed in the present invention such as, for example, chemi-activated (e.g. enzymes and metal salts) including, by way of example and not limitation, other substances and compositions that can produce singlet oxygen with or, less preferably, without activation by an external light source. Thus, for example, molybdate (MoO₄⁼) salts and chloroperoxidase and myeloperoxidase plus bromide or chloride ion have been shown to catalyze the conversion of hydrogen peroxide to singlet oxygen and water. For the above examples of sensitizers, hydrogen peroxide may be included as an ancillary reagent, chloroperoxidase may be bound to a surface and molybdate may be incorporated in the aqueous phase of a liposome, respectively. Other sensitizers included within the scope of the invention are compounds that are not true sensitizers but which on excitation by heat, light, ionizing radiation, or chemical activation will release a molecule of singlet oxygen. The best known members of this class of compounds include the endoperoxides, such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Further sensitizers are disclosed in the following references: Di Mascio et al., FEBS Lett., 355: 287 (1994) (peroxidases and oxygenases); Kanofsky, J. Biol. Chem. 258: 5991-5993 (1983) (lactoperoxidase); Pierlot et al., Meth. Enzymol., 319: 3-20 (2000) (thermal lysis of endoperoxides); and the like.

Attachment of a binding agent, such as an antibody, to the cleavage-inducing moiety may be direct or indirect, covalent or non-covalent, and can be accomplished by well-known techniques, commonly available in the literature. See, for example, “Immobilized Enzymes,” Ichiro Chibata, Halsted Press, New York (1978); Cuatrecasas, J. Biol. Chem. 245:3059 (1970). A wide variety of functional groups are available or can be incorporated. Functional groups include carboxylic acids, aldehydes, amino groups, cyano groups, ethylene groups, hydroxyl groups, mercapto groups, and the like. The manner of linking a wide variety of compounds is well known and is amply illustrated in the literature (see above). The length of a linking group to a binding agent may vary widely, depending upon the nature of the compound being linked, the effect of the distance on the specific binding properties, and the like.

It may be desirable to have multiple cleavage-inducing moieties attached to a binding agent to increase, for example, the number of active species generated. In one approach, the binding agent has a plurality of sites for attachment such as, for example, an antibody, a lectin, and so forth. To further enhance the number of cleavage-inducing moieties, a hub molecule or nucleus is employed. The hub nucleus is a polyfunctional material, normally polymeric, having a plurality of functional groups, e.g., hydroxy, amino, mercapto, carboxy, ethylenic, aldehyde, etc., as sites for linking. An exemplary hub material is aminodextran, which may be attached to binding agents, such as antibodies, using well-known techniques. Preferably, NHS-esters of cleavage-inducing moieties are then reacted with the aminodextran for attachment.

Photosensitizers as Cleavage-Inducing Moieties

As mentioned above, the preferred cleavage-inducing moiety in accordance with the present invention is a photosensitizer that produces singlet oxygen. As used herein, “photosensitizer” refers to a light-adsorbing molecule that when activated by light converts molecular oxygen into singlet oxygen. Photosensitizers may be attached directly or indirectly, via covalent or non-covalent linkages, to the binding agent of a class-specific reagent. Guidance for constructing of such compositions, particularly for antibodies as binding agents, available in the literature, e.g. in the fields of photodynamic therapy, immunodiagnostics, and the like. The following are exemplary references: Ullman, et al., Proc. Natl. Acad. Sci. USA 91, 5426-5430 (1994); Strong et al., Ann. New York Acad. Sci., 745: 297-320 (1994); Yarmush et al., Crit. Rev. Therapeutic Drug Carrier Syst., 10: 197-252 (1993); Pease et al., U.S. Pat. No. 5,709,994; Ullman et al., U.S. Pat. No. 5,340,716; Ullman et al., U.S. Pat. No. 6,251,581; McCapra, U.S. Pat. No. 5,516,636; and the like.

Likewise, there is guidance in the literature regarding the properties and selection of photosensitizers suitable for use in the present invention. The following are exemplary references: Wasserman and R. W. Murray. Singlet Oxygen. (Academic Press, New York, 1979); Baumstark, Singlet Oxygen, Vol. 2 (CRC Press Inc., Boca Raton, Fla. 1983); and Turro, Modern Molecular Photochemistry (University Science Books, 1991).

The photosensitizers are sensitizers for generation of singlet oxygen by excitation with light. The photosensitizers include dyes and aromatic compounds, and are usually compounds comprised of covalently bonded atoms, usually with multiple conjugated double or triple bonds. The compounds typically absorb light in the wavelength range of about 200 to about 1,100 nm, usually, about 300 to about 1,000 nm, preferably, about 450 to about 950 nm, with an extinction coefficient at its absorbance maximum greater than about 500 M⁻¹ cm⁻¹, preferably, about 5,000 M⁻¹ cm⁻¹, more preferably, about 50,000 M⁻¹ cm⁻¹, at the excitation wavelength. The lifetime of an excited state produced following absorption of light in the absence of oxygen will usually be at least about 100 nanoseconds, preferably, at least about 1 millisecond. In general, the lifetime must be sufficiently long to permit cleavage of a linkage in a reagent in accordance with the present invention. Such a reagent is normally present at concentrations as discussed below. The photosensitizer excited state usually has a different spin quantum number (S) than its ground state and is usually a triplet (S=1) when the ground state, as is usually the case, is a singlet (S=0). Preferably, the photosensitizer has a high intersystem crossing yield. That is, photoexcitation of a photosensitizer usually produces a triplet state with an efficiency of at least about 10%, desirably at least about 40%, preferably greater than about 80%.

Photosensitizers chosen are relatively photostable and, preferably, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they may have extended aromatic structures.

A large variety of light sources are available to photoactivate photosensitizers to generate singlet oxygen. Both polychromatic and monochromatic sources may be used as long as the source is sufficiently intense to produce enough singlet oxygen in a practical time duration. The length of the irradiation is dependent on the nature of the photosensitizer, the nature of the cleavable linkage, the power of the source of irradiation, and its distance from the sample, and so forth. In general, the period for irradiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and not limitation, lasers such as, e.g., helium-neon lasers, argon lasers, YAG lasers, He/Cd lasers, and ruby lasers; photodiodes; mercury, sodium and xenon vapor lamps; incandescent lamps such as, e.g., tungsten and tungsten/halogen; flashlamps; and the like.

Examples of photosensitizers that may be utilized in the present invention are those that have the above properties and are enumerated in the following references: Turro, Modern Molecular Photochemistry (cited above); Singh and Ullman, U.S. Pat. No. 5,536,834; Li et al., U.S. Pat. No. 5,763,602; Ullman, et al., Proc. Natl. Acad. Sci. USA 91, 5426-5430 (1994); Strong et al., Ann. New York Acad. Sci., 745: 297-320 (1994); Martin et al., Methods Enzymol., 186: 635-645 (1990); Yarmush et al., Crit. Rev. Therapeutic Drug Carrier Syst., 10: 197-252 (1993); Pease et al., U.S. Pat. No. 5,709,994; Ullman et al., U.S. Pat. No. 5,340,716; Ullman et al., U.S. Pat. No. 6,251,581; McCapra, U.S. Pat. No. 5,516,636; Wohrle, Chimia, 45: 307-310 (1991); Thetford, EP 0484027; Sessler et al., SPIE, 1426: 318-329 (1991); Madison et al., Brain Research, 522: 90-98 (1990); Polo et al., Inorganica Chimica Acta, 192: 1-3 (1992); Demas et al., J. Macromol. Sci., A25: 1189-1214(1988); and the like. Exemplary photosensitizers are listed in Table 2.

TABLE 2
Exemplary Photosensitizers

| | |
|---|---|
| Hypocrellin A | Tetraphenylporphyrin |
| Hypocrellin B | Halogenated rhodamine derivatives |
| Hypericin | metallo-Porphyrins |
| Halogenated fluorescein derivatives | Phthalocyanines |
| Rose bengal | Naphthalocyanines |
| Merocyanine 540 | Texaphyrin-type macrocycles |
| Methylene blue | Hematophorphyrin |
| 9-Thioxanthone | 9,10-Dibromoanthracene |
| Chlorophylls | Benzophenone |
| Phenaleone | Chlorin e6 |
| Protoporphyrin | Perylene |
| Benzoporphryin A monacid | Benzoporphryin B monacid |

Photosensitizers may also be incorporated in, for example, a latex particle, to form photosensitizer beads, e.g. as disclosed by Pease et al., U.S. Pat. No. 5,709,994. For this purpose, the photosensitizer is preferably relatively non-polar, to assure dissolution into the lipophilic member. For example, the photosensitizer rose bengal is covalently attached to 0.5 micron latex beads by means of chloromethyl groups on the latex to provide an ester linking group, as described in J. Amer. Chem. Soc. 97:3741 (1975).

V. Synthesis of G-L-E Compounds

The compounds of the invention comprise the lipophilic group (G), the cleavable linkage (L), and the electrophoretic group (E), as described above. The compounds of the present invention, having the structure G-L-E, can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 3^{rd} Ed., Vols. A and B (Plenum 1992), and Green and Wuts, PROTECTIVE GROUPS IN ORGANIC SYNTHESIS 2^{nd} Ed. (Wiley 1991). Starting materials for the compounds of the invention may be obtained using standard techniques and commercially available precursor materials, such as those available from Aldrich Chemical Co. (Milwaukee, Wis.), Sigma Chemical Co. (St. Louis, Mo.), Lancaster Synthesis (Windham, N.H.), Apin Chemicals, Ltd. (New Brunswick, N.J.), Ryan Scientific (Columbia, S.C.), Maybridge (Cornwall, England) and Trans World Chemicals (Rockville, Md.).

The procedures described herein for synthesizing the compounds of the invention may include one or more steps of protection and deprotection (e.g., the formation and removal of acetal groups). See, for example, the synthesis of three exemplary membrane binding electrophoretic probes in Example 1, below. In addition, the synthetic procedures can include various purifications, such as column chromatography, flash chromatography, thin-layer chromatography (TLC), recrystallization, distillation, high-pressure liquid chromatography (HPLC) and the like. Also, various techniques well known in the chemical arts for the identification and quantification of chemical reaction products, such as proton and carbon-13 nuclear magnetic resonance (¹H and ¹³C NMR), infrared and ultraviolet spectroscopy (IR and UV), X-ray crystallography, elemental analysis (EA), HPLC and mass spectroscopy (MS) can be used as well. Methods of protection and deprotection, purification and identification and quantification are well known in the chemical arts.

The chemistry for performing the types of syntheses to form the charge-imparting moiety or mobility modifier as a peptide chain is well known in the art. See, for example, Marglin et al., Ann. Rev. Biochem. 39:841-866 (1970). In general, such syntheses involve blocking, with an appropriate protecting group, those functional groups that are not to be involved in the reaction. The free functional groups are then reacted to form the desired linkages. The peptide can be produced on a resin as in the Merrifield synthesis (Merrifield, J. Am. Chem. Soc. (1980) 85:2149-2154 and Houghten et al., Int. J. Pep. Prot. Res. (1980) 16:311-320). The peptide is then removed from the resin according to known techniques.

A summary of the many techniques available for the synthesis of peptides may be found in J. M. Stewart et al., “Solid Phase Peptide Synthesis, W.H. Freeman Co, San Francisco (1969); and J. Meienhofer, “Hormonal Proteins and Peptides”, (1973), vol. 2, p. 46, Academic Press (New York), for solid phase peptide synthesis; and E. Schroder et al., “The Peptides”, vol. 1, Academic Press (New York), 1965 for solution synthesis.

For synthesis of electrophoretic tag probes employing phosphoramidite, or related chemistry, many guides are available in the literature: Handbook of Molecular Probes and Research Products, 8^{th} edition (Molecular Probes, Inc., Eugene, Oreg., 2002); Beaucage and Iyer, Tetrahedron, 48: 2223-2311 (1992); Molko et al., U.S. Pat. No. 4,980,460; Koster et al., U.S. Pat. No. 4,725,677; Caruthers et al., U.S. Pat. Nos. 4,415,732; 4,458,066; and 4,973,679; and the like. Many of these chemistries allow components of the electrophoretic probe to be conveniently synthesized on an automated DNA synthesizer, e.g. an Applied Biosystems, Inc. (Foster City, Calif.) model 392 or 394 DNA/RNA Synthesizer, or the like.

Synthesis of electrophoretic tag reagents comprising nucleotides as part of the mobility-modifying moiety can be easily and effectively achieved via assembly on a solid phase support using standard phosphoramidite chemistries. The resulting mobility modifying moiety may be linked to the label and/or polypeptide-binding moiety as discussed above.

The aforementioned label conjugates with different electrophoretic mobility permit a multiplexed detection of multiple polypeptides having induced binding sites. It is, of course, within the purview of the present invention to prepare any number of label conjugates for performing multiplexed determinations.

VI. Antibodies

In general, an antibody is an immunoglobulin (a class of globular protein) present in the serum of an animal that is produced by lymphocytes (plasma cells) in response to the presence of an antigen. An antibody specifically binds to, and is thereby defined as complementary with, a particular spatial and polar organization of another molecule. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained.

Antibodies can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies.

A “monoclonal antibody” (MAB) is an immunoglobulin produced by a single clone of lymphocytes, i.e. the progeny of a single B cell, which recognizes only a single epitope on an antigen. MAB can be produced experimentally from hybridoma cells, e.g. according to the standard techniques of Köhler and Milstein, Nature 265:495-497, 1975. Reviews of monoclonal antibody techniques are found in Lymphocyte Hybridomas, ed. Melchers et al., Springer-Verlag (New York 1978); Nature 266: 495 (1977); Science 208: 692 (1980), and Methods of Enzymology 73 (Part B): 3-46 (1981).

Monoclonal antibodies can be targeted against cell surface receptors, e.g. epidermal growth receptor receptor (EGFR), as therapeutic candidates. Accordingly, cells carrying a particular receptor, such as a cancer-specific receptor, are injected into a mouse, thereby inducing the mouse's antibody producing cells (B lymphocytes) to produce antibodies against all of the receptors on the cell surface. These B cells are extracted and fused to immortalized (endlessly replicating) cells in culture to create hybridomas. Modifications of this standard method have been developed which create “humanized” MAB. Thus, in one embodiment of the invention, test monoclonal antibodies are produced by antibody-secreting hybridoma cells, and the test antibodies added to the target cells are harvested from a culture of the hybridoma cells.

MAB can also be produced by a library of phage engineered to display a library of immunoglobulin fragments as binding moieties on the phage surface, and the test antibodies added to the target cells are phage particles. Briefly, to produce a phage display antibody library, cDNAs of immunoglobulins isolated by PCR from immunized B lymphocytes (each cell of which makes antibodies against only one antigen) are inserted into the genome of filamentous phage or a phagemid vector which is introduced into E. coli host cells. As the phages replicate, antibodies encoded by the genes of the various B lymphocytes are expressed on their surfaces. The phage library is screened to identify those phage whose displayed proteins (antibodies) bind to a target.

In another approach for the preparation of antibodies, a sequence coding for antibody binding site(s) can be excised from chromosomal DNA and inserted into a cloning vector, which can be expressed in bacteria to produce recombinant proteins having the corresponding antibody binding sites. To facilitate extraction or purification of an expressed antibody from an expression system, a gene sequence encoding a defined affinty peptide tag (e.g. 6×His, HA, myc, etc.) can be inserted at the amino or carboxy-terminus of the immunoglobulin gene sequence.

Various conventional methods exist for isolation and purification of the monoclonal antibodies from other proteins and other contaminants (see Köhler and Milstein, supra). In general, antibodies can be purified by known techniques such as chromatography, e.g., DEAE chromatography, ABx chromatography, and the like, filtration, and so forth.

“Secondary antibodies” are antibodies that recognize the common portion, typically the Fc region, of the primary screening monoclonal antibodies. They are typically species specific, as the protein sequence and carbohydrates modifications in the Fc region are conserved within the species. These anti-immunoglobulin (IgG) are generated by immunizing animals with purified IgG from another animal. The secondary antibodies can also specifically recognize different antibody isotypes, which are determined by one of several “constant” gene fragments being used in assembling the antibodies. Many secondary antibodies are commercially available for research or product development.

VII. The Membrane

The membranes for use in the practice of the invention can be obtained from cells, such as a cellular membrane, nuclear membrane, mitochondrial membrane, or other intracellular membrane, or can be artificially created, as exemplified by micelles and liposomes. The cell(s) used in the methods described herein can be of any origin, including from prokaryotes, eukaryotes, or archeons, but preferably contain membranes that are lipophilic. The cell(s) may be living or dead. If obtained from a multicellular organism, the cell may be of any cell type. Thus, the cell(s) may be a cultured cell line or a primary isolate, the cell(s) may be mammalian, amphibian, reptilian, plant, yeast, bacterium, spirochetes, or protozoan. The cell(s) may be, for example, human, murine, rat, hamster, chicken, quail, goat or dog. The cell may be a normal cell, a mutated cell, a genetically manipulated cell, a tumor cell, hybridomas that are positive for secretion of selected antibodies, and the like. Of particular interest are membranes obtained from the type of cell that differentially expresses (over-expresses or under-expresses) a disease-causing gene. As is apparent to one skilled in the art, various cell lines, such as CHO, for example, may be obtained from public or private repositories. The largest depository agent is American Type Culture Collection, which offers a diverse collection of well-characterized cell lines derived from a vast number of organisms and tissue samples.

Exemplary cell types from multicellular organisms include acidophils, acinar cells, pinealocytes, adipocytes, ameloblasts, astrocytes, basal (stem) cells, basophils, hepatocytes, neurons, bulging surface cells, C cells, cardiac muscle cells, centroacinar cells, chief cells, chondrocytes, Clara cells, columnar epithelial cells, corpus luteal cells, decidual cells, dendrites, endrocrine cells, endothelial cells, enteroendocrine cells, eosinophils, erythrocytes, extraglomerular mesangial cells, fetal fibroblasts, fetal red blood cells, fibroblasts, follicular cells, ganglion cells, giant Betz cells, goblet cells, hair cells, inner hair cells, type I hair cells, hepatocytes, endothelial cells, Leydig cells, lipocytes, liver parenchymal cells, lymphocytes, lysozyme-secreting cells, macrophages, mast cells, megakaryocytes, melanocytes, mesangial cells, monocytes, myoepithelial cells, myoid cells, neck mucous cells, nerve cells, neutrophils, oligodendrocytes, oocytes, osteoblasts, osteochondroclasts, osteoclasts, osteocytes, pillar cells, sulcal cells, parathyroid cells, parietal cells, pepsinogen-secreting cells, pericytes, pinealocytes, pituicytes, plasma cells, platelets, podocytes, spermatocytes, Purkinje cells, pyramidal cells, red blood cells, reticulocytes, Schwann cells, Sertoli cells, columnar cells, skeletal muscle cells, smooth muscle cells, somatostatin cells, enteroendocrine cells, spermatids, spermatogonias, spermatozoas, stellate cells, supporting Deiter cells, support Hansen cells, surface cells, surface epithelial cells, surface mucous cells, sweat gland cells, T lymphocytes, theca lutein cells, thymocytes, thymus epithelial cell, thyroid cells, transitional epithelial cells, type I pneumonocytes, and type II pneumonocytes.

Cell membranes can also be obtained from a cell type that is associated with a particular disease or with a specific disease stage. The association with a particular disease or disease stage may be established by the cell's aberrant behavior in one or more biological processes such as cell cycle regulation, cell differentiation, apoptosis, chemotaxsis, cell motility and cytoskeletal rearrangement. A disease cell may also be confirmed by the presence of a pathogen causing the disease of concern (e.g. HIV for AIDS and HBV for hepatitis B). The types of diseases involving abnormal functioning of specific types of cells may include but are not limited to autoimmune diseases, cancer, obesity, hypertension, diabetes, neuronal and/or muscular degenerative diseases, cardiac diseases, endocrine disorders, and any combinations thereof. Exemplary types of tumor cells include adenomas, carcinomas, adenocarcinomas, fibroadenomas, ameloblastomas, astrocytomas, mesotheliomas, cholangiocarcinomas, cholangiofibromas, cholangiomas, chondromas, chondrosarcomas, chordomas, choriocarcinomas, craniopharyngiomas, cystadenocarcinomas, cystadenomas, dysgerminomas, ependymomas, epitheliomas, erythroid leukemias, fibroadenomas, fibromas, fibrosarcomas, gangliogliomas, ganglioneuromas, ganglioneuroblastomas, gliomas, granulocytic leukemias, hemangiomas, hemangiopericytomas, hemangiosarcomas, hibemomas, histiocytomas, keratoacanthomas, leiomyomas, leiomyosarcomas, lipomas, liposarcomas, luteomas, lymphangiomas, lymphangiosarcomas, lymphomas, medulloblastomas, melanomas, meningiomas, mesotheliomas, myelolipomas, nephroblastomas, neuroblastomas, neuromyoblastomas, odontomas, oligodendrogliomas, osteochondromas, osteomas, osteosarcomas, papillomas, paragangliomas, pheochromocytomas, pinealomas, pituicytomas, retinoblastomas, rhabdomyosarcomas, sarcomas, schwannomas, seminomas, teratomas, thecomas and thymomas.

In another aspect of the invention, the membrane comprises liposomes. “Liposomes” are self-assembling structures comprising one or more lipid bilayers. Liposomes are usually composed of phospholipid bilayers, although other molecules, such as cholesterol or fatty acids can also be included in the bilayer construction. The phospholipid constituents of liposomes includes a hydrophobic lipid tail connected to a head constructed of various glycerylphophate or silicone derivatives. Liposomes are thus normally made from amphipathic lipids comprise a polar (hydrophilic) headgroup region covalently linked to one or two non-polar (hydrophobic) acyl chains. Energetically unfavorable contacts between the hydrophobic acyl chains and the aqueous medium are generally believed to induce lipid molecules to rearrange such that the polar headgroups are oriented towards the aqueous medium while the acyl chains reorient towards the interior of the bilayer. An energetically stable structure is formed in which the acyl chains are effectively shielded from coming into contact with the aqueous medium. The hydrophobic interaction between the fatty acid tails thus creates the liposomal bilayers in aqueous solutions. In more complicated liposomal structures, one or more of the lipid bilayers can surround an aqueous compartment and comprises two opposing monolayers of amphipathic lipid molecules. Liposomes are thus completely closed bilayer membranes containing an encapsulated aqueous phase. Thus, liposomes may be any variety of multilamellar vesicles (concentric membrane bilayers each separated by an aqueous layer) or unilamellar vesicles (possessing a single membrane bilayer).

The liposomes may be prepared according to the method of Bangham et al., J. Mol. Biol. 13:238-252 (1965), in which phospholipids are suspended in an organic solvent which is then evaporated to dryness, leaving a waxy deposit of phospholipid on the reaction vessel. An appropriate amount of aqueous medium is added, the mixture is allowed to “swell,” and the resulting liposomes, consisting of multilamellar vesicles, are dispersed by mechanical means. The structure of the resulting membrane bilayer is such that the hydrophobic (non-polar) “tails” of the lipid orient toward the center of the bilayer while the hydrophilic (polar) “heads” orient towards the aqueous phase. This technique provided the basis for the development of the small sonicated unilamellar vesicles described by Papahadjopoulos and Miller, Biochim. Biophys. Acta. 135:624-638 (1967). Normally, mixtures of phospholipids in aqueous solution will spontaneously associate to form liposomal structures, although techniques for controlling the size and shape of the liposomes are known in the art.

VIII. Labeling Membranes with G-L-E Moieties

The compounds of the invention, having the structure G-(L-E)ₘ, can be attached to cells, liposomes, etc. by incorporation into the membrane bilayer. Without being bound by theory, the G-(L-E)ₘ compounds can be incorporated into the lipid membranes in an orientation and manner similar to that of phospholipids, where the hydrophobic moiety, comprising the hydrocarbon chains, is oriented inward, and the more hydrophilic entities L-E are oriented outwards. Thus, as in the usual cellular membranes, the hydrocarbon portion of the inventive compounds can be incorporated into the lipid environment while the hydrophilic L-E can be exposed to the aqueous interface at the membrane surface. The compounds of the invention can thus be incorporated into the membranes such that the cleavable linkage and the electrophoretic moiety are exposed on the surface of the membrane and are accessible to the cleavage reagent, such as singlet oxygen, described above. The compounds of the invention can also be used to label liposomes. Useful liposomes include cationic phospholipids, neutral phospholipids, lipids and mixtures thereof.

Where intact cellular structures are required, the methods used to label the cells preferably cause minimal disruption of the cell and of the integrity of membranes. In addition, the cells can be fixed and treated with routine histochemical or cytochemical procedures, where the procedure preferably does not affect the labeling. Additional components may be included, such as targeting peptides or proteins, fusion peptides (e.g., from Sendai virus, influenza virus, hemagluttinating virus of Japan (HVJ)), envelope proteins of viruses, polycationic substances such as poly-L-lysine or DEAE-dextran, molecules which bind to the surface of airway epithelial cells including antibodies, adhesion molecules and growth factors, and the like.

The membranes can be labeled with the compounds of the invention according to the method described in Barak and Webb, J. Cell Biol. 90:595-604 (1981). Typically, the membrane, such as the intact cell, is contacted with the compounds of the invention, preferably in an aqueous medium. The aqueous medium can be water, an organic solvent, such as DMSO, DMF, DMA, or a mixture thereof, and can contain buffers such as phosphate, acetate, tris, and the like. The membranes and compounds having the structure G-L-E are contacted for between 1 min. to about 1 week, preferably about 1 h to 76 h, more preferably about 2 h to about 48 h, or any integer in between. The formulations may additionally be subjected to chemical or mechanical treatment, such as the addition of a surfactant (Tween 80, for example), shaking, stirring, electroporation, and the like. Alternatively, the formulation can be heated to about 30° C. to 50° C., preferably about 35° C. to about 40° C., until labeling is achieved. After labeling, the unbound components can be removed by washing, or by centrifugation, for example, and the labeled membranes isolated.

IX. General Procedures for Assays Employing Electrophoretic Probes

The following general discussion of methods and examples of specific assays are by way of illustration and not limitation. One skilled in the art will be able to apply the technology herein in assaying for a variety of analytes in many assay formats known to the skilled artisan. An exemplary protocol for a multiplex binding assay, using a direct binding, heterogeneous format, is described in Example 4.

Conventional buffers may be used, such as phosphate, carbonate, HEPES, MOPS, Tris, borate, etc., as well as other conventional additives, such as salts, stabilizers, organic solvents, etc. The aqueous medium may be solely water or may include from 0.01 to 80 or more volume percent of a co-solvent.

The combined assay components are incubated for a time and at a temperature that permit a substantial number of binding events to occur. Generally, the time for incubation after combination of all or a portion of the reagents is at least 5 min, more usually at least 15 min, before irradiating the mixture or adding further reagents. Low to moderate temperatures are normally employed for the incubation, to avoid leaching of lipid-anchored probes from the cell surfaces. Incubation temperatures will normally range from about 4° C. to about 10° C.

Once a solution of cleaved e-tag reporters is prepared and is free of any interfering components, the composition of the solution is analyzed. The released tags from an assay are preferably separated on a single separation medium or format, meaning that a sample mixture containing the combined tags is applied to a single separation medium, such as electrophoretic separation medium, a chromatography medium, or a mass spectroscopy medium, and all of the sample product/substrates components are separated on that medium.

A preferred separation medium is an electrophoretic medium. The analysis may employ capillary electrophoresis devices, microfluidic devices or other devices that can separate a plurality of compounds electrophoretically, providing resolved bands of the individual e-tag reporters. A preferred separation device is a microfluidics device of the type described above for separating charged components across a separation channel, according to well-known methods. The electrophoretic device is generally connected to a data processor for receiving and processing data from the device, as well as operating the electrophoretic device. Electrophoretic separation and band resolution of a plurality of probes and substrates is readily accomplished by this method.

Conveniently, an aliquot, generally not more than about 5 μl, is transferred to the sample reservoir of a microfluidics device or capillary electrophoretic device, either directly through electrophoretic or pneumatic injection into an integrated system or by syringe, capillary or the like. Microfluidics devices are described in a number of domestic and foreign Letters Patent and published patent applications. See, for example, U.S. Pat. Nos. 5,750,015; 5,900,130; 6,007,690; and WO 98/45693; WO 99/19717 and WO 99/15876. The conditions under which the separation is performed are conventional and will vary with the nature of the products. Longer times will be required for products that have similar mobilities under the conditions of the electrophoresis.

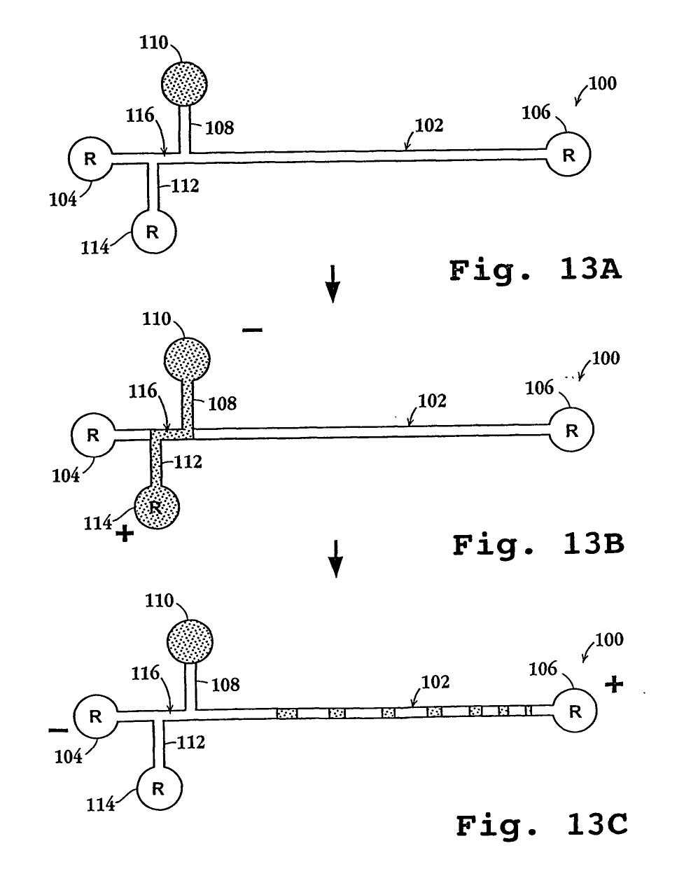

By way of illustration, FIG. 13A shows a microchannel network 100 in a microfluidics device of the type detailed in the application noted above, for sample loading and electrophoretic separation of a sample of probes and tags produced in the assay above. Briefly, the network includes a main separation channel 102 terminating at upstream and downstream reservoirs 104, 106, respectively. The main channel is intersected at offset axial positions by a side channel 108 that terminates at a reservoir 110, and a side channel 112 that terminates at a reservoir 114. The offset between the two side channel forms a sample loading zone 116 within the main channel.

In operation, the assay mixture from above is placed in sample reservoir 110, illustrated in FIG. 13A. As noted, the assay mixture contains one or more target cells with surface-bound electrophoretic probe, one or more test ligands, and optionally, an eTag standard. The assay reaction, involving initial ligand binding to target cell(s), followed by cleavage of probe linkers, may be carried out in sample reservoir 110, or alternatively, the assay reactions can be carried out in another reaction vessel, with the reacted sample components the added to the sample reservoir.

To load released eTags into the sample-loading zone, an electric field is applied across reservoirs 110, 114, as indicated in the Figure, drawing negatively charged released probes from reservoir 110 into loading zone 116 (FIG. 13B), while uncharged or positively charged sample components remain in the sample reservoir. The released tags in the loading zone can now be separated by conventional capillary electrophoresis (FIG. 13C), by applying an electric filed across reservoirs 104, 106, as indicated in the Figure.

From the resulting electrophoretic pattern, the tags, and corresponding cell types labeled by the tags, can be identified. This is typically done by placing a fluorescence detector near the downstream end of the separation channel, and constructing a electropherogram, of the type shown schematically in FIG. 1A, of the separated eTag components, first to determine the separation characteristic (in this case, electrophoretic mobility) as above, and secondly, to measure signal intensity, e.g., peak height or peak area, as a measure of the relative amount of tag associated with each probe. Methods for detecting and quantifying levels of a detectable probe are well known. In one preferred method, the tags are fluorescent labeled. A standard fluorescence-emission source is directed against a detection zone in a downstream portion of the separation medium, and fluorescence emission of the zone is measured by a standard light detector. The signal height or area recorded provides a measure of product and substrate concentration in the sample.

Addition of a known quantity of a control fluorophore to each sample before separation of the e-tag reporters by electrophoresis allows conversion of relative fluorescent signals into absolute quantities. Any fluorophore that does not interfere with detection of the e-tag reporter signals can be used for normalizing the fluorescent signal. The control signal will preferably have an electrophoretic mobility that is different from that of any of the e-tag reporters in the sample, and may have the same or a different emission wavelength. Exemplary control fluorescent molecules include ROX, FAM, and fluorescein.

With the above detection information, it is now possible to assign each detected tag to a particular cell type, and to compare the relative levels of each detected tag, as a measure of ligand binding to that cell.

X. Applications

The assays described herein are useful for screening of multiple cell lines or populations in one experiment, in place of primary and secondary screens. Applications include screening for cell-specific binders and for antagonists for such binders. The assays provide various advantages over current screening methods, in which cell types are screened individually, using a fluorescently labeled test compound, and the presence or absence of the test compound is determined, in a separate run for each cell type, by FACS sorting. The present assay formats are high throughput and automation compatible and allow multiple cell lines and/or or test compounds to be screened simultaneously. The assays also have a much higher sensitivity than conventional methods, in that unique receptors having a density as low as 100-1000 per cell may be detected.

The methods described herein can be used, for example, in typing cell samples based on the composite expression pattern of cell surface antigens, as recognized by a multiplexed set of binders such as monoclonal antibodies, ligands, and pharmacological agents. Using the methods described herein, the differential expression of cell surface antigens among multiple cell samples can be analyzed and compared within a single reaction. Antibody screening can be used to identify novel cell surface markers that are specific for disease phenotypes, based on the isolation of specific antibodies, in particular, monoclonal antibodies from culture supernatants of hybridoma libraries. Examples of such cell surface markers include receptors for physiologic ligands (e.g. secreted proteins or peptides, lipids, lipoproteins, metabolites, glycosylated macromolecules, cognitive or adhesion counter-receptors on another cell surface, microbial antigens, non-mammalian toxins) and antigenic non-receptor specific epitopes (e.g. peptide sequences, carbohydrate epitopes).

In particular, the multiplex assays can be used for cancer cell typing or subtyping. Cancer cells are clonal in origin, and can be typed based on detailed characterization of cell markers, gene expression patterns, chromosomal translocations, cytogenetic features, gene mutations and methylations, as well as tissue source. These various signatures of cancers can potentially correlate with disease etiology, progress, and prognosis. The signatures can provide a set of markers to facilitate the development of pharmacogenomics databases, tracking drug response based on cancer types or subtypes. The ability to type or subtype cancer samples can also be used to appropriately group patients for clinical management and research studies.

In a method of cancer typing or subtyping using multiplex monoclonal antibody screening methods described above, eTag reporters can be used to label live cell samples of cancer patients for screening against a panel of monoclonal antibodies for targets with known or unknown biologic functions. When conducted in a reasonably large-scale study containing a number of monoclonal antibodies and cancer cell samples, clustered patterns of antibody reactivity can be used to divide the cancer cell samples into groups. Comparison of the clinical characteristics within each group of cancer samples will further facilitate the typing or subtyping of cancers.

Monoclonal antibodies have also rapidly emerged as drugs of choice against a number of diseases. In particular, monoclonal antibodies directed against tumor-associated antigens expressed on the tumor cell surface have found application in the immunodiagnosis and immunotherapy of human tumors. In particular, MAB targeted to the epidermal growth factor receptor (EGFR) are of great interest, due to the high level of expression of this receptor in patients with solid tumors. MAB have also been developed which target molecules implicated in other diseases, such as autoimmune diseases, AIDS, asthma, and other inflammatory disorders. For example, a MAB directed against CD11a, a protein on the surface of T-lymphocytes, is being developed by Genentech for treatment of psoraisis. Other groups are developing monoclonal antibodies targeting DC18, another T-lymphocyte protein involved in inflammatory responses and in tissue damage following heart attack, and against tumor necrosis factor (TNF), also involved in inflammation.

The present methods can be used for high-throughput screening of candidate antibodies against multiple cell types, for targeting selected cell types for therapeutic or diagnostic purposes. Normal cells and diseased cells can be screened simultaneously to evaluate the specificity of the molecule for the targeted cell type.

XI. Experimental

Below are examples of specific embodiments for carrying out the present invention. The examples are offered for illustrative purposes only, and are not intended to limit the scope of the present invention in any way.

### EXAMPLE 1

### Synthesis of eTags with Lipid Anchors

A. “Pro28-amide” (see FIG. 11A)

Reaction of 5-carboxyfluorescein with N-hydroxysuccinimide (NHS) and 1,3-dicyclohexylcarbodiimide (DCC) in DMF gave the corresponding NHS ester, which was then treated with ethylenediamine. The resulting amine was reacted with α-bromophenylacetic acid NHS ester to afford the desired α-bromo derivative 1 (see FIG. 11A). Treatment of 1 with 11-mercaptoundecanoic acid and ET₃N in DMF provided the acid 2. Finally, conversion of 2 to its NHS ester followed by reaction with dioctadecylamine gave the target structure Pro28-amide, also referred to herein as Pro28.

B. “Pro29-amide” (see FIG. 11B)

Reaction of 5-aminofluorescein with α-bromophenylacetyl chloride (prepared by treating α-bromophenylacetic acid with oxalyl chloride) gave the bromo compound 3 (see FIG. 11B), which was then reacted with 3-mercaptopropanoic acid and triethylamine in DMF. The resulting α-thioacid 4 was finally converted, as described above, to Pro29-amide.

C. “Pro36-amide” (see FIG. 11C)

The synthesis of Pro36-amide, utilizing 5-carboxyfluorescein as starting material, was carried out as follows (see FIG. 11C). The fluorescein derivative was condensed with 1,10-diaminodecane, forming an amide linkage at the less hindered carboxyl group. The terminal amine was reacted with the NHS ester of Fmoc-protected glycine. The Fmoc-protected amine was then deprotected and reacted with the NHS ester of α-bromophenylacetic acid. Nucleophilic displacement of the bromide with 3-mercaptopropanoic acid, followed by NHS activation of the acid and condensation with dioctadecylamine, gave the product.

### EXAMPLE 2

### Conjugation of Streptavidin with Al-Phthalocyanine Tetrasulfonic Acid

To 3 mL of 17 mg/mL streptavidin in 33 mM Borax was added 65 μL of 46 mg/mL (42 nmol/μL) of Al-Phthalocyanine tetrasulfonic acid hexanoate NHS ester in DMF. The mixture was incubated for 16 hours in the dark at 4° C. Separation of conjugated and non-conjugated Al-Phthalocyanine tetrasulfonic acid (PcS) was performed on 1.5×46 cm Sepharose CL-6B equilibrated in 1×PBS. Analysis of the produce showed that about 3 PcS (sensitizer) molecules were conjugated per streptavidin molecule (using A₂₈₀=3.0 for 1 mg/mL StAv and ε=180,000 for PcS).

### EXAMPLE 3A

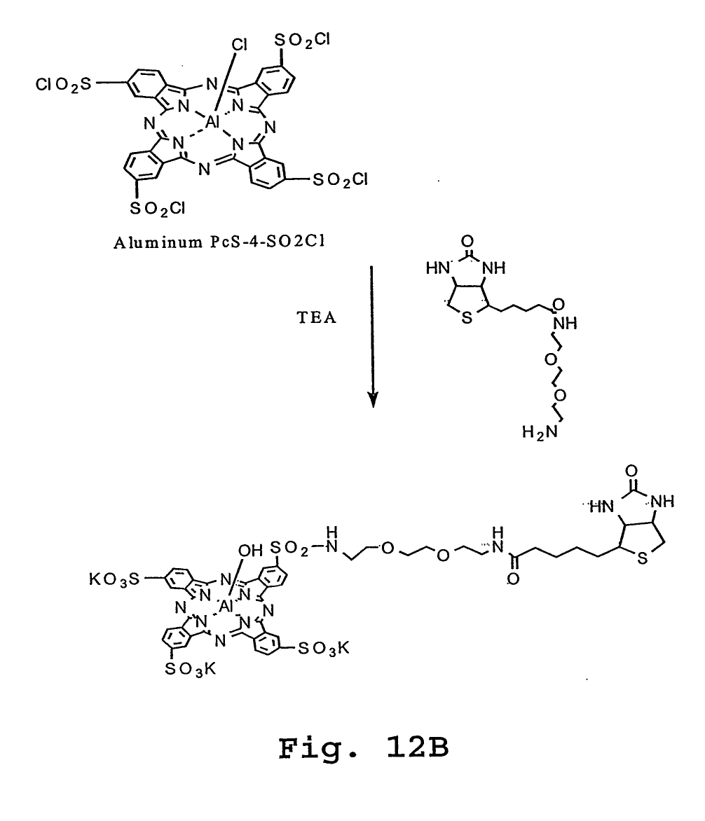

### Linking of Photosensitizer to Des-Biotin Derivative (FIG. 12A)

To a dry 100 mL round bottom flask fitted with a stir bar was added aluminum phthalocyanine tetrasulfonyl chloride (150 mg, 0.15 mmol). Anhydrous THF (10 mL) was added to dissolve the compound. Desthiobiotin amine (50 mg, 0.16 mmol), dissolved in 0.5 mL of DMF, was added to the reaction flask, followed by triethylamine (0.2 mL). The reaction was stirred at room temperature for 16 hrs, then quenched with potassium carbonate solution (1 mL, 10% solution). Stirring was continued for 1 hour. Solvent was removed, and the product was purified by reverse phase column chromatography, eluting with acetonitrile-water (20%). Homogeneous fractions were pooled, and the solvent was removed to obtain the desthiobiotinylated phthalocyanine (FIG. 12A) (Al PcS-DES Biotin; 14.2 mg). TLC: rev. phase, Rf, 0.5 (acetonitrile, water, 5:3)

### EXAMPLE 3B

### Linking of Photosensitizer to PEO-Biotin Derivative (FIG. 12B)

To a dry 100 mL round bottom flask fitted with a stir bar was added aluminum phthalocyanine tetrasulfonyl chloride (96 mg, 0.1 mmol) and anhydrous DMF (2 mL) to dissolve. EZ link biotin-PEO-amine (Pierce Chemical Co.) (50 mg, 0.13 mmol), dissolved in 1 mL THF and 3 mL DMF, was added to the reaction flask, followed by triethylamine (0.1 mL). The reaction flask was covered with aluminum foil and the contents stirred at room temperature for 16 hrs. The reaction was quenched with potassium carbonate solution (1 mL, 10% solution) and stirring continued for 1 hour. Solvent was removed, and the product was purified by reverse phase column chromatography, eluting with acetonitrile-water (20%). (The column was wrapped with aluminum foil to protect it from light). Homogeneous fractions were pooled and the solvent removed to obtain the biotinylated phthalocyanine (FIG. 12B) (15.3 mg, 13.3%). TLC: rev. phase, Rf, 0.5 (acetonitrile, water, 5:3)

### EXAMPLE 4

### Exemplary Protocol for Multiplex Antibody Screen Assay

(The intermittent cell washing steps can be eliminated in streamlined and optimized protocols.)

1. Cell samples are separately aliquoted at 10⁴ to 10⁶ cells per 96-well on round-bottom microplates. Culture media are removed followed centrifugation at 1,200 rpm for 5 min to pellet cells. (Alternatively, when membrane filter plates are used for automation, the centrifugation wash step can be substituted with controlled vacuum suction and continuous liquid dispension on a robotic platform. The following steps refer to the centrifugation wash format.)

2. To cell pellets, 10 μl of 10-100 μM lipophilic eTag in culture media is added for incubation at 37° C., 5% CO₂ for 15-30 min. Different lipophilic eTags are added to different cell pellets in separate wells.

3. eTag-labeled cells are cooled to 4° C. for about 10 min and washed twice to remove unbound eTags.

4. Each lipophilic eTag-labeled cell sample is resuspended in 10 μl ice-cold culture media, and aliquots of 5 μl or less of each are mixed together. (Alternatively, in a large scale screen, different cell samples can be labeled with lipophilic eTags in batches before mixing.)

5. Biotin-labeled receptor-specific antibody is added to the labeled cell sample mixture in 2-10 μl of culture media. A predetermined saturating antibody concentration should be used in the final reaction. Alternatively, for a hybridoma screen, mixed cell samples are pelleted, and 10-20 μl of diluted hybridoma supernatant is added.

6. Assay mixture is incubated at 4° C. for 1 hr.

7. For a hybridoma screen, biotinylated secondary antibody is incubated and washed as above.

8. The cells are washed once or twice by centrifugation and addition of 200 μl of culture media.

9. Solubilized or bead-immobilized Streptavidin-photosensitizer conjugate in 10 μl culture media is added to cells and incubated at 4° C. for 15 min.

10. The cells are washed twice by centrifugation and addition of 200 μl of culture media.

11. After the last wash, the cell pellets are resuspended in 15-20 μl of 10 mM Tris-HCl (pH 7.5) containing 10 pM 5-FAM and 100 pM fluorescein reference standards.

12. Samples are illuminated for 5 min at >600 nm wavelength.

13. Aliquots of the supernatants are transferred to 96-wells on a MicroAmp Optical reaction plate.

14. Samples are analyzed by CE analysis, e.g. on an ABI-3100 or MegeBACE 1000 analyzer.

### EXAMPLE 5

### Loading Electrophoretic Probe into Cell Membranes

Approximately 10⁵ HL-60 cells were incubated for 15 min at 37° C. in culture media (RPMI-1640 with 10% fetal calf serum) with various concentrations of electrophoretic probe Pro28 (shown in FIG. 11A), after which the cells were twice washed by centrifugation and resuspension in culture media (200 μL in each wash) at 4° C. to remove unincorporated probe. The labeled cells were incubated in culture media with 10 μg/mL anti-HLA-A,B,C antibody (e.g., W6/32 monoclonal antibody, available from eBioscience (San Diego, Calif.), or like antibody) for 1 hour at 4° C. with shaking in a reaction volume of 10 μL, after which unbound antibody was removed by washing, as described above. Cells were then resuspended in 10 μL culture media and incubated for 1 hour at 4° C. with biotinylated goat anti-mouse IgG (at 4 μg/mL, e.g. available from Zymed Laboratories (South San Francisco, Calif.)), after which the cells were again washed as described above. Cells were resuspended in 10 μL culture media, and 5 μL of avidinated photosensitizer bead mix was added, after which the mixture was incubated for 20 min at 4° C. (Photosensitizer bead mix consists of 1 μg/5 μL of photosensitizer beads in culture media containing 1 μM 2-(4-hydroxy azobenzene)benzoic acid (HABA), which enhances signal by preventing nonspecific binding of eTags to avidin. Photosensitizer beads are disclosed in U.S. Pat. Nos. 5,709,994; and 6,346,384, which are incorporated by reference; and in J. Am. Chem. Soc. 97: 3741 (1975); and like references. Such beads, including streptavidinated photosensitizer beads, can be obtained, for example, from Packard Biosciences, Meriden, Conn.) Cells were then washed three times by centrifugation and resuspension in culture media (200 μL in each wash) at 4° C., followed by resuspension in 15 μL of electrophoresis buffer. Electrophoresis buffer is an aqueous solution of 10 mM Tris-HCl (pH 7.5) containing 10 pM 5-FAM and 200 pM fluorescein as CE separation standards. The resuspended cells were illuminated at 680 nm for 10 min using an LED Illuminator (model OD-1399, Opto Diode Corp., Newbury Park, Calif.) to release eTags, after which 7.5 μL was mixed with 7.5 μL of 10 mM Tris-HCl (pH 7.5) in a well of a MicroAmp CE analysis plate (Applied Biosystems, Inc., Foster City, Calif.) for electrophoretic separation. Released eTags were separated with an Applied Biosystems, Inc. model 3100 16-channel capillary electrophoresis instrument (Pop 4 capillaries, run temperature 45° C., run time 30 min, 60 sec injection at 3000 v).

FIG. 3 shows that the signal from released eTags (peak area in relative fluorescent units (RFU)) varies linearly with the concentration of Pro28 over a range of from about 20 to 75 μM, and that above about 75 μM the signal no longer increases with increasing probe concentration, suggesting an upper limit to the amount of probe that can be loaded into the membrane.

### EXAMPLE 6

### Concentrations of Photosensitizer-Linked Antibody for Cleaving Membrane-Anchored Electrophoretic Probes

In this example, 10⁵ HL-60 cells and 10⁵ Jurkat cells were separately labeled with Pro28 (FIG. 11A), using the procedure of Example 5 with 30 μM Pro28, and then incubated with varying concentrations of photosensitizer-linked antibody specific for cell membrane targets. Specifically, in 10 μL reaction volumes, anti-HLA-A,B,C antibody (for HL-60 cells) or anti-CD81 antibody (for Jurkat cells) were added at various concentrations, as shown in FIGS. 4A-B, and incubated in culture media for 1 hour at 4° C. The cells were washed twice as described in Example 5, and then incubated with biotinylated goat anti-mouse IgG secondary antibody, as described in Example 5; after which they were combined with avidinated photosensitizer bead mix and illuminated, as described in Example 5, to release eTags. The released eTags were analyzed as described above, and the results are shown in FIGS. 4A-B. Under these conditions, as little as 0.4 μg/mL photosensitizer-linked antibody was able to detect eTag labeled cells.

### EXAMPLE 7

### Detection of eTag-Labeled Cells in Mixed Cell Populations Based on Release of eTags

In this example, cells labeled with membrane-anchored electrophoretic probes (“eTag labeled cells”) were detected in mixed populations containing the eTag labeled cells and unlabeled cells. HL-60 cells and Jurkat cells were separately labeled with Pro28 as described above, after which mixed populations of cells were formed with labeled HL-60 cells and unlabeled EL-4 cells, and separately with labeled Jurkat cells and unlabeled EL-4 cells. In every case, the total number of cells in the mixed population was 10⁵; that is, the mixed populations differed only in number of labeled cells that they contained, which varied from 10³ to 10⁵ (i.e. the latter being the case with no mixing). Each mixed population of cells was treated and released eTags analyzed as described above, where anti-HLA-A,B,C was used as the primary antibody against HL-60 cells and anti-CD81 was used as the primary antibody against Jurkat cells. FIGS. 5-6 show that in the two cases, cells making up as few as 1% of a mixed population can be detected with membrane-anchored electrophoretic probes.

It is claimed:

1. A method of determining the binding specificity of a ligand to a cell surface moiety, the method comprising the steps of:
(a) providing one or more cell types such that each cell type has a different membrane-anchored electrophoretic probe, each membrane-anchored electrophoretic probe having a membrane anchoring moiety connected by a cleavable linkage to an electrophoretic tag, said tag having distinct optical or electrophoretic properties with respect to electrophoretic tags of other cell types;
(b) combining the one or more cell types with a ligand, and
(c) exposing the cell types and ligand to conditions such that at least one cleavable linkage is cleaved in at least one membrane-anchored electrophoretic probe of cells having a cell surface moiety to which said ligand is bound, whereby one or more electrophoretic tags are released; and
(d) electrophoretically separating and identifying the one or more released electrophoretic tags, to determine the specificity of the ligand for the cell surface moieties of the cell types.

2. The method of claim 1, wherein said exposing comprises
attaching to each cell having a bound ligand, a proximity-dependent cleavage inducing group, such that each said probe is cleavable only by such a cleavage inducing group on the same cell surface as the probe, and
activating said cleavage inducing group.

3. The method of claim 2, wherein said probe is cleavable by a short-lived chemical species generated by a sensitizer group, and said exposing includes
attaching such a sensitizer group to each cell having a bound ligand, and
activating the sensitizer group.

4. The method of claim 3, wherein the sensitizer group is a photosensitizer.

5. The method of claim 3, wherein the sensitizer group is attached to the ligand.

6. The method of claim 3, wherein the sensitizer group is attached to a secondary molecule which forms a complex with the ligand.

7. The method of claim 6, wherein the ligand is an antibody, the sensitizer is conjugated to a secondary antibody immunospecific against the antibody, and said exposing includes adding the secondary antibody and conjugated sensitizer to the cells and bound antibody.

8. The method of claim 1, wherein said step of combining comprises isolating cell types having at least one ligand bound to at least one cell surface moiety.

9. The method of claim 8, wherein said cleavable linkage is chemically cleavable, photochemically cleavable, or enzymatically cleavable.

10. A method of identifying a cell surface antigen specific to substantially only one of a plurality of cell types, the method comprising the steps of:
(a) providing a plurality of cell types with cell surface antigens such that each cell type has a different membrane-anchored electrophoretic probe, the membrane-anchored electrophoretic probe having a membrane anchoring moiety connected by a cleavable linkage to an electrophoretic tag, said tag having distinct optical or electrophoretic properties with respect to electrophoretic tags of other cell types of the plurality;
(b) combining the one or more cell types with a candidate antibody;
(c) exposing the cell types and antibody to conditions such that at least one cleavable linkage is cleaved in at least one membrane-anchored electrophoretic probe of cells having a cell surface antigen to which said antibody is bound, whereby one or more electrophoretic tags are released;
(d) electrophoretically separating and determining the relative quantities of the one or more released electrophoretic tags to determine whether the candidate antibody binds to a cell surface antigen present on substantially only one of the plurality of cell types; and
(e) repeating steps (b)-(d) until a cell surface antigen specific to substantially only one of the plurality of cell types is identified.

11. The method of claim 10, wherein said exposing comprises
attaching to each cell having a bound antibody, a proximity-dependent cleavage inducing group, such that each said probe is cleavable only by such a cleavage inducing group on the same cell surface as the probe, and
activating said cleavage inducing group.

12. The method of claim 11, wherein said probe is cleavable by a short-lived chemical species generated by a sensitizer group, and said exposing includes
attaching such a sensitizer group to each cell having a bound antibody, and
activating the sensitizer group.

13. The method of claim 12, wherein the sensitizer group is a photosensitizer.

14. The method of claim 13, wherein the sensitizer group is attached to the antibody.

15. The method of claim 13, wherein the sensitizer is conjugated to a secondary antibody immunospecific against the antibody, and said exposing includes adding the secondary antibody and conjugated sensitizer to the cells and bound antibody.

16. The method of claim 10, wherein said step of combining comprises isolating cell types having at least one antibody bound to at least one cell surface antigen.

17. The method of claim 16, wherein said cleavable linkage is chemically cleavable, photochemically cleavable, or enzymatically cleavable.

18. The method of claim 10, wherein the determining of step (d) comprises measuring the area of tag peaks in an electropherogram of said released tags, and identifying a cell surface antigen specific to substantially only one of the plurality of cell types comprises identifying one test tag peak in said electropherogram that is at least 90% of the sum of the areas of all the test tag peaks in the electropherogram.

19. The method of claim 18, wherein said one test eTag peak is at least 95% of the sum of the areas of all the test eTag peaks in the electropherogram.

20. The method of claim 18, wherein identifying a cell surface antigen specific to substantially only one of the plurality of cell types comprises identifying one test tag peak in said electropherogram having an area that is at least twice the area of the next largest test tag peak in the electropherogram.

21. The method of claim 20, wherein said one test tag peak is at least four times the area of the next largest test tag peak in the electropherogram.

22. The method of claim 10, further comprising identifying said candidate antibody which binds to said cell surface antigen.

23. A method of determining the binding affinity of a compound for a cell surface antigen, the method comprising the steps of:
(a) providing one or more test cell-antibody pairs, each such pair comprising (i) a test cell having a membrane-anchored electrophoretic probe, the membrane-anchored electrophoretic probe having a membrane anchoring moiety connected by a cleavable linkage to an electrophoretic tag, said tag having distinct optical or electrophoretic properties with respect to electrophoretic tags of other test cells of the plurality, and (ii) at least one antibody specific for an cell surface antigen of the test cell of such pair, such cell surface antigen being different from cell surface antigens on other test cells of the plurality;
(b) combining the compound with the test cell-antibody pairs under conditions that permit the binding of the antibodies and the compound to one or more cell surface antigens recognized by said compound or antibodies;
(c) exposing the test cell-antibody pairs to conditions such that at least one cleavable linkage is cleaved in at least one membrane-anchored electrophoretic probe of cells having a cell surface antigen to which said antibody is bound, whereby one or more electrophoretic tags are released;
(d) electrophoretically separating the released tags; and
(e) determining the relative quantities of each of the one or more released electrophoretic tags to determine the binding affinity of the compound for the cell surface antigens.

24. The method of claim 23, wherein said exposing comprises
attaching to each cell having a bound antibody, a proximity-dependent cleavage inducing group, such that each said probe is cleavable only by such a cleavage inducing group on the same cell surface as the probe, and
activating said cleavage inducing group.

25. The method of claim 24, wherein said probe is cleavable by a short-lived chemical species generated by a sensitizer group, and said exposing includes
attaching such a sensitizer group to each cell having a bound antibody, and
activating the sensitizer group.

26. The method of claim 25, wherein the sensitizer group is a photosensitizer.

27. The method of claim 26, wherein the sensitizer group is attached to the antibody.

28. The method of claim 26, wherein the sensitizer is conjugated to a secondary antibody immunospecific against the antibody, and said exposing includes adding the secondary antibody and conjugated sensitizer to the cells and bound antibody.

29. The method of claim 23, further comprising the step of comparing the relative quantities obtained in step (e) with those obtained when steps (a), (c) and (d) are carried out in the absence of step (b).

30. A method of determining the binding specificity of a compound for an internalizing cell surface receptor, the method comprising the steps of:
(a) providing a plurality of test cell-antibody pairs, each such pair comprising (i) a test cell having a membrane-anchored electrophoretic probe, the membrane-anchored electrophoretic probe having a membrane anchoring moiety connected by a cleavable linkage to an electrophoretic tag having distinct optical or electrophoretic properties with respect to electrophoretic tags of other test cells of the plurality, and (ii) at least one antibody effective to bind to an internalizing cell surface receptor of the test cell of such pair;
(b) combining the compound with the plurality of test cells under conditions that permit the endocytosis of complexes that form between the compound and one or more of the internalizing cell surface receptors;
(c) combining with the test cells the plurality of antibodies under conditions that permit binding to internalizing cell surface receptors;
(d) exposing the test cell-antibody pairs to conditions such that at least one cleavable linkage is cleaved in at least one membrane-anchored electrophoretic probe of cells having an internalizing cell surface receptor to which said antibody is bound, whereby one or more electrophoretic tags are released;
(e) electrophoretically separating the released tags; and
(f) determining the relative quantities of each of the one or more released electrophoretic tags to determine the specificity of the compound for the plurality endocytosing cell surface receptors.

31. The method of claim 30, wherein said exposing comprises
attaching to each cell having a bound antibody, a proximity-dependent cleavage inducing group, such that each said probe is cleavable only by such a cleavage inducing group on the same cell surface as the probe, and
activating said cleavage inducing group.

32. The method of claim 30, wherein the internalized cell surface receptor of each test cell is different from internalizing cell surface receptors on other test cells of the plurality.

33. The method of claim 30, wherein said probe is cleavable by a short-lived chemical species generated by a sensitizer group, and said exposing includes
attaching such a sensitizer group to each cell having a bound antibody, and
activating the sensitizer group.

34. The method of claim 33, wherein the sensitizer group is a photosensitizer.

35. The method of claim 33, wherein the sensitizer group is attached to the antibody.

36. The method of claim 33, wherein the sensitizer is conjugated to a secondary antibody immunospecific against the antibody, and said exposing includes adding the secondary antibody and conjugated sensitizer to the cells and bound antibody.

37. The method of claim 30, further comprising the step of comparing the relative quantities obtained in step (f) with those obtained when steps (a) and (c)-(e) are carried out in the absence of step (b).
